(19) United States
(12) Patent Application Publication
(10) Pub. No.: US 2002/0081588 A1
(43) Pub. Date: Jun. 27, 2002

(54) Multi-sensor array for electrochemical recognition of nucleotide sequences and methods

(75) Inventors: Thierry De Lumley-woodyear, Paris (FR); Daren J. Caruana, London (GB); Adam Heller, Austin, TX (US)

Correspondence Address: MERCHANT & GOULD PC, P.O. BOX 2903, MINNEAPOLIS, MN, 55402-0903, US

(73) Assignee: TheraSense, Inc., Alameda, CA (US)

(21) Appl. No.: 09/746,620

(22) Filed: Dec. 21, 2000

Related U.S. Application Data
(63) Continuation of application No. PCT/US99/14460, filed on Jun. 24, 1999.
(60) Provisional application No. 60/090,517, filed on Jun. 24, 1998.
(60) Provisional application No. 60/093,100, filed on Jul. 16, 1998.
(60) Provisional application No. 60/114,919, filed on Jan. 5, 1999.

(51) Int. Cl. C12Q 1/68; C12M 1/34

(52) U.S. Cl. 435/6; 435/287.2

(57) ABSTRACT

Sensor and method of its production and use in the electrochemical detection of nucleic acid sequences.

### FIELD OF THE INVENTION

The present invention is generally related to a microelectrode for the analysis of nucleic acid sequences, including recognition and determination of specific diagnostic oligonucleotides. More particularly, the invention relates to electrodes and to arrays of microelectrodes useful for detection and recognition of oligonucleotides, and to a novel method of reproducibly producing electrodes and microelectrode arrays by reactive electrophoretic deposition of chemically activated nucleotides and/or oligonucleotides.

### BACKGROUND OF THE INVENTION

Detection of oligonucleotide pairing, e.g., DNA or RNA sensing, has a variety of applications, including, for example, sequencing of the human genome, detection of disease, including detecting specific disease-inducing agents such as microorganisms or mutants leading to specific types of cancer, and identification of bioconjugation-blocking drugs. Typically, detection of a specific nucleotide sequence requires amplification of a nucleic acid sample, as the number of copies of an oligonucleotide needed to generate a signal useful for rapid detection exceeds 10 million, and is in the order of more than one billion for known analytical methods. Furthermore, conventional sequencing techniques are still time consuming, despite increasing automation, e.g., polymerase chain reaction (PCR) methods.

To address these problems, arrays of DNA and RNA sensors are being developed using tools for the production of integrated circuits. These arrays require fewer copies of a sequence for detection, and permit scanning of a large variety of sequences in a shortened time period.

DNA-sensing silicon chips (2.5×2.5 cm) with arrays of 35×35 micrometers (1.5×10⁻⁵ cm²) pixels, each pixel having a different, short (up to 10 bases) oligonucleotide sequence, are now available. The massive number of sequences required on this chip is synthesized on these arrays nucleotide-by-nucleotide, using simple, known techniques, for example, using multiple photochemical or photolithographic process steps. These process steps are very time-consuming. In addition, use of these arrays to identify a particular nucleic acid sequence through optical and/or spectroscopic means requires the presence of not fewer than 10⁹ copies on each pixel. These arrays and methods are not well adapted for ease of manufacture or for effective probing of a very small chip having a maximum of sensing pixels, their size approaching the size and density of electronic devices constituting functional elements of integrated circuits.

Electrochemical means for recognition of oligonucleotide-hybridization using macroelectrodes has been described. Millan et.al. (1994 Anal. Chemistry 66:2943) detected voltammetrically the intercalation of redox couples in double strands of DNA and succeeded in observing the mutation associated with cystic fibrosis. Xu et. al. (1994 J. Am. Chem. Soc. 116:8386; 1995 J Am. Chem. Soc. 117:2627) observed hybridization through the electrogenerated chermiluminescence of a metal chelate tag after its intercalation in double stranded DNA. Korri-Youssoufi et.al. (1997 J. Am. Chem. Soc. 119:7388) measured the increase in the resistivity of electrochemically co-polymerized pyrrole and oligonucleotide-substituted pyrrole upon hybridization. Such co-polymerization resulted in a high (10⁻⁶ mol cm²) coverage by the single-stranded oligonucleotide. At 10 μM concentration of the oligonucleotide, 6×10⁸ hybridization events per cm² were detected.

Measures of the relative advantages of different oligonucleotide sensing systems include size, the number of copies detected, the selectivity (assessed by the ability to sense mutations), and the ease of manufacture and cost of the system. There is no accepted figure of merit of the combination of these measures. In some applications, particularly those relevant to sensing in combinatorial arrays where the samples are small, the combination of the size of the sensing element, the number of copies detected, and the ability to differentiate between long oligonucleotide sequences differing in a single base are particularly relevant factors. The size of the detecting elements defines their surface density in the array; the number of copies detected defines the number of amplification (e.g., PCR) cycles needed to detect a particular sequence, the error rate increasing with the number of cycles; and the ability to differentiate between oligonucleotides of increasing length, differing only in a single base, defines the balance between the ability to locate mutants and the specificity.

Millan et al., Anal. Chem., 1994, 66:2943; Singahal et al., Anal. Chem., 1997, 69:4828; and Napier et al., Bioconjugate Chem., 1997, 8:906 have shown that electrochemical techniques are well suited for measuring hybridization events. A desired yet unapproached goal is to accurately detect a single base mutation in a single copy of a gene, using a sensing element the dimensions of which are not different from that of the smallest gate in an integrated circuit. The use of microelectrodes has proven successful in numerous applications where miniaturization was important. (Kawagoe, et al., Anal. Chem., 1991, 63:2961; Pishko et al., Anal. Chem., 1992, 63:2668; Abe et al., Anal. Chem., 1992, 64:2160). Frequently, however, particularly in arrays where signals of different elements were compared, it was not the electrode size that prevented the use of micron-sized electrodes, but the reproducibility of their specificity-providing coatings.

Routes to selectively coat electrodes have been described. Korri-Youssoufi et al., electrochemically copolymerized oligonucleotide modified monomers of pyrrole (Am. Chem. Soc., 1997, 119:7388) then constructed an array of 48 microelectrodes of 50 μm×50 μm, each derivatized with a different oligonucleotide sequence. Electrochemical means for recognition of oligonucleotide hybridization using microelectrodes are described by Heller et al. (U.S. Pat. Nos. 5,605,662; 5,632,957; 5,849,486) and Hill et al. (U.S. Pat. No. 4,840,893). With the above, hybridization was analyzed through measuring the fluorescence of a hybrid-bound molecule (Korri-Youssoufi et al.; Heller et al.) or through a decrease in measured steady state current resulting from competitive inhibition of hybridization by target DNA (Hill et al.).

The need for rapid, efficient DNA-detection methods is great, particularly in view of the vast amount of information stored in genetic material and becoming available for rapid processing. Accordingly, there is a need for new arrays and methods of forming the arrays with improved sensitivity, ease of use, reduced time of probing per element, and verification of the information. Progress toward this goal would be made by the simultaneous reduction of electrode size and reduction of the number of copies required for detection, e.g., detecting a single base mismatch in an oligonucleotide.

### SUMMARY OF THE INVENTION

In the present invention, an electrode, that can be a microelectrode array suitable for efficient and rapid detection and recognition of low copy numbers of nucleic acid sequences is provided. The inventive electrodes and microelectrodes are produced by electrophoretically depositing individual nucleic acid molecules upon individual microelectrodes, thus providing, without the requirement of a photolithographic mask, an element in an array of individually addressable, hybridization and/or melting-sensing elements. The deposited sensor oligonucleotides are coupled to a redox polymer, which redox polymer is disposed on the electrode. A redox polymer disposed on the electrodes provides the basis for electrochemical detection of hybridization events.

The microelectrode of the invention is reproducibly activated for the detection of hybridization of an oligonucleotide at a low copy number (e.g., about 40,000 copies) through the reactive electrophoretic deposition of a chemically activated oligonucleotide. Hybridization of a complimentary copy and/or the melting of the hybrid, is electrochemically, preferably amperometrically, observed with an individual microelectrode (about 1-10 micrometers (8×10⁻⁷ cm²) diameter). By controlling the stringency of hybridization conditions, such as temperature, oligonucleotides having a single base pair mismatch can be discriminated by the electrodes of the invention.

The electrode arrays of the invention are particularly useful for the analysis and detection of a specific nucleic acid sequence diagnostic of a particular disease. For example, the small copy number required of the instant electrochemical hybridization-detection system permits screening of nucleic acid sequences contained in a drop of blood for the presence of a nucleic acid sequence diagnostic of a specific microorganism, including bacterial, fungal, and eukaryotic pathogens. The electrochemical detection system of the invention also permits rapid and efficient detection of mutant nucleic acid sequences diagnostic of specific diseases, including cancer screens, with the ability to discriminate a single base-pair mismatch in a small copy number sample. Using the sensor arrays of the invention, the sample nucleic acid need not be amplified, but can be directly used without amplification as obtained from the tissue sample.

The electrode arrays of the invention may also be useful for the simultaneous but multiple analysis and detection of nucleic acid sequences diagnostic of one or more particular diseases. For example, the multisensor array may have a flow-channel system arranged so that the sample passes over multiple sensors, each specific for a particular nucleic acid sequence. Target DNA sequences contained within the sample hybridize to the diagnostic sensors and are thus captured and directly detected.

The above summary of the present invention is not intended to describe each disclosed embodiment or every implementation of the present invention. The Figures and the detailed description which follow more particularly exemplify these embodiments.

### BRIEF DESCRIPTION OF THE DRAWINGS

The invention may be more completely understood in consideration of the following detailed description of various embodiments of the invention in connection with the accompanying drawings, in which:

FIG. 1 is a plan diagram of a DNA-sensing microelectrode array of the invention.

FIG. 2 is a diagram shown in cross-section and depicting a microelectrode array having attached single-stranded oligonucleotide.

FIG. 3 is a diagram shown in cross-section and depicting a microelectrode array having attached hybridized sensor oligonucleotide and labeled probe oligonucleotide.

FIG. 4 is a graph showing potential-time transients upon electrophoretic deposition of (a) the redox polymer and (b) the oligonucleotide. Currents of 20 μA for 2147 s and −10 μA for 900 s were passed, respectively.

FIG. 5 is a graph showing cyclic voltammograms for 10 μm microelectrodes electrophoretically coated with (a) redox polymer, (b) redox polymer and oligonucleotide, and (c) redox polymer and oligonucleotide after hybridization in a high ionic strength solution. Scan rate was 50 mV/s; voltammograms started at 0.3 mV vs. Ag/AgCl.

FIG. 6 is a graph showing scan rate dependence of the oxidation (squares) and reduction (circles) peak currents of the redox polymer on a microelectrode in buffer. The solid line is a linear regression fit for the reduction peak current and the broken line is a best fit for the oxidation peak current.

FIG. 7 is a graph showing amperometric response of a typical coated microelectrode with redox polymer and oligonucleotide, hybridized with (a) pd(A)₂₅₋₃₀-HRP and (b) pd(G)₁₈₋₂₀-HRP. Hydrogen peroxide (1 mM) was added to the 5 mL test solution to initiate the reaction. The background current was between −5 and −12 pA. It took about 200 seconds for the current to attain a stable baseline after a potential of 0.00V vs Ag/AgCl was applied. The response time was governed by the mixing of the hydrogen peroxide in the solution. The arrow shows the point at which H₂O₂ was introduced into the cell.

FIG. 8 is a graph showing the loss of current upon melting of the hybrid: time and temperature dependence of the electrocatalytic hydrogen peroxide reduction current upon ramping the temperature linearly (broken line) from 20° C. to 60° C. at a rate of 0.25° C./minute. The vertical arrow shows the point at which hydrogen peroxide was introduced into the cell.

FIGS. 9A-9I are a series of graphs showing hybridization or non-hybridization to a target oligonucleotide by complementary strand (FIGS. 9A, 9D, 9G); single base pair mismatch (FIGS. 9B, 9E, 9H); and four base pair mismatch (FIGS. 9C, 9F, 9I) under hybridization conditions of 25° C. (FIGS. 9A-C); 45° C. (FIGS. 9D-F); and 57° C. (FIGS. 9G-I).

FIG. 10 is a schematic representation of the nucleic acid hybridization assay on the electrodes of the invention, showing electrode 10; redox polymer 12; sensor oligonucleotide 14; probe oligonucleotide 16; detection marker 18.

FIG. 11 is a graph showing the fifth cyclic voltammograms of electrophoretically, deposited redox polymer (solid line) and redox polymer reacted with oligonucleotide probe (dashed line), using the sensor described in Example 7. (7 μm diameter carbon microelectrode; scan rate 50 mV s⁻¹; pH 7 HEPES buffer containing 1 M NaCl and 1.0 mM EDTA).

FIG. 12 is a graph showing the effect of probe oligonucleotide loading on the hybridization of target as measured by change in catalytic current. Probe loading was achieved by duration of electrophoretic deposition: 1 minute (a), 2.5 minutes (b), 5 minutes (c), and 10 minutes (d) (stirred, 1 mL pH 7 HEPES, 1.0 M NaCl. buffer with 1.0 mM EDTA, 1.0 mM H₂O₂; −0.06 V (Ag/AgCl). 10 μl of the 40 nM SBP-labeled target solution was added at 0 seconds).

FIGS. 13A-13C are graphs showing an increase in the catalytic current of microelectrodes at 25° C. (FIG. 13A), 45° C. (FIG. 13B) and 57° C. (FIG. 13C) after adding the SBP-labeled target fully complementary or partially mismatched targets for perfectly matched target (curve a); target with a single mismatched base (curve b); target with four mismatched bases (curve c) (10μl of the 40 nM SBP-labeled target solution were added at 0 seconds; stirred 1 mL pH 7 HEPES buffer, 1 M NaCl with 1.0 mM H₂O₂; −0.06 V (Ag/AgCl)). The dashed lines represent the best fit of the data to Equation 4.

FIG. 14 is a graph showing current-time plot of the catalytic current of a microelectrode coated with the probe-bearing redox polymer. The SBP-labeled target with four mismatched bases was introduced at 550 seconds (arrow A), followed by introduction of the SBP-labeled perfectly matching target at 1450 seconds (arrow B) (stirred 1 μL pH 7 HEPES buffer, 1 M NaCl, with 1.0 mM H₂O₂; −0.06 V (Ag/AgCl) thermostatted at 45° C.).

FIGS. 15A-15C are schematic diagrams of the detection system of the invention. Probe oligonucleotides are covalently bound to the electron-conducting redox polymer on the microelectrode. Upon hybridization of labeled target nucleic acid sequences, electrical contact is established between the SBP-heme centers and the electrode via the redox polymer. This contact enables the electrocatalytic reduction of H₂O₂ to water through the cycle shown in FIG. 15 B. Hybridization is thereby translated to current of H₂O₂ electroreduction. FIG. 15C is a schematic diagram of a preferred embodiment of the invention demonstrating electrochemical detection of hybridization of a target nucleic acid sequence to an immobilized first probe and to a second, labeled probe.

FIG. 16 is a schematic diagram of a preferred detection system of the invention. A nucleotide sequence, for example, a fragment of a gene or of RNA, is permitted to react with one or more oligonucleotide probes. A first oligonucleotide probe is immobilized on the electrode via the redox polymer. A second oligonucleotide probe is labeled, preferably with thermostable peroxidase. The second, labeled probe may be immobilized or free. Hybridization of a target sequence at both the first and second probes results in measured current.

While the invention is amenable to various modifications and alternative forms, specifics thereof have been shown by way of example in the drawings and will be described in detail. It should be understood, however, that the intention is not to limit the invention to the particular embodiments described. On the contrary, the intention is to cover all modifications, equivalents, and alternatives falling within the spirit and scope of the invention.

### DETAILED DESCRIPTION OF THE PREFERRED EMBODIMENT

The present invention is applicable to the sensing of nucleic acid hybridizations, for example, during nucleic acid synthesis, analysis of unknown nucleic acid sequences, diagnosis of altered, e.g., mutant, nucleic acid sequences, and the like. In particular, the present invention is directed to an apparatus, method of manufacture, and method of use of a microelectrode array formed by electrophoretic deposition of individual activated oligonucleotides on individual microelectrodes. While the present invention is not so limited, an appreciation of various aspects of the invention will be gained through a discussion of the examples provided below.

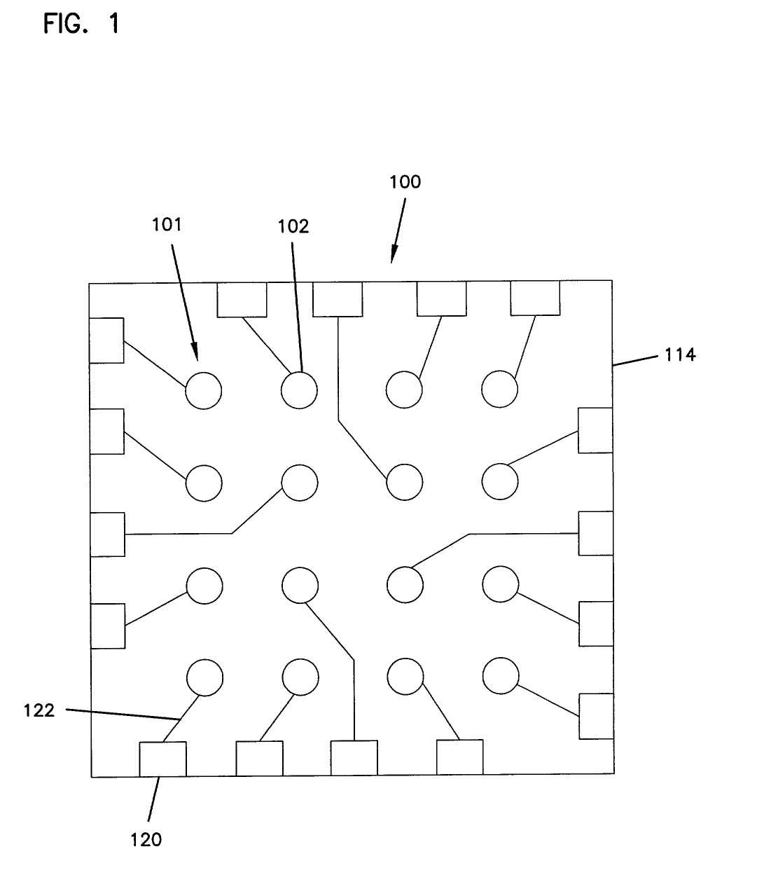

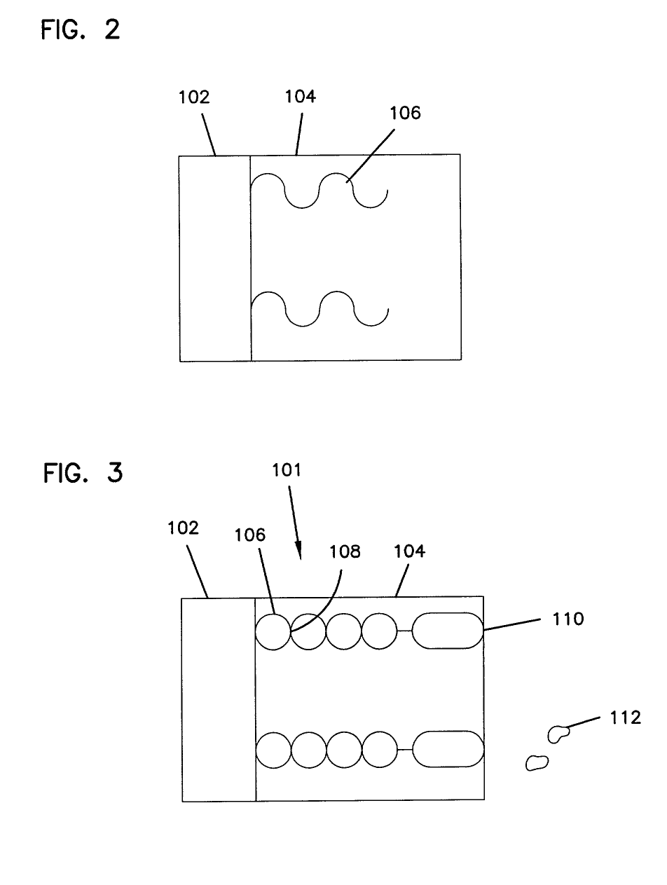

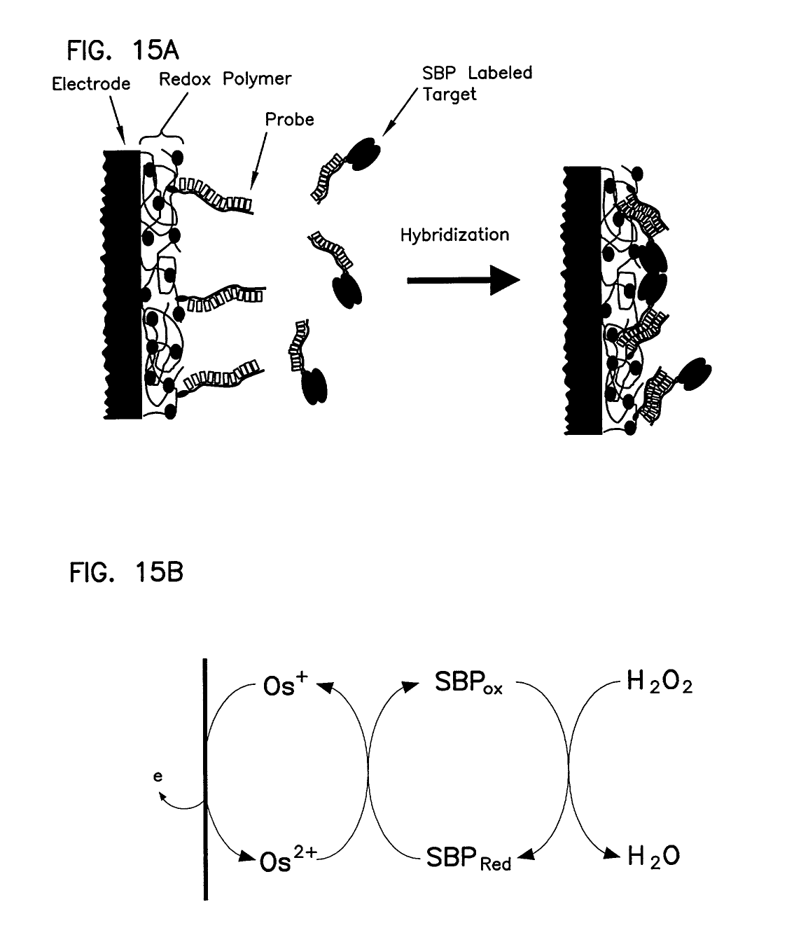

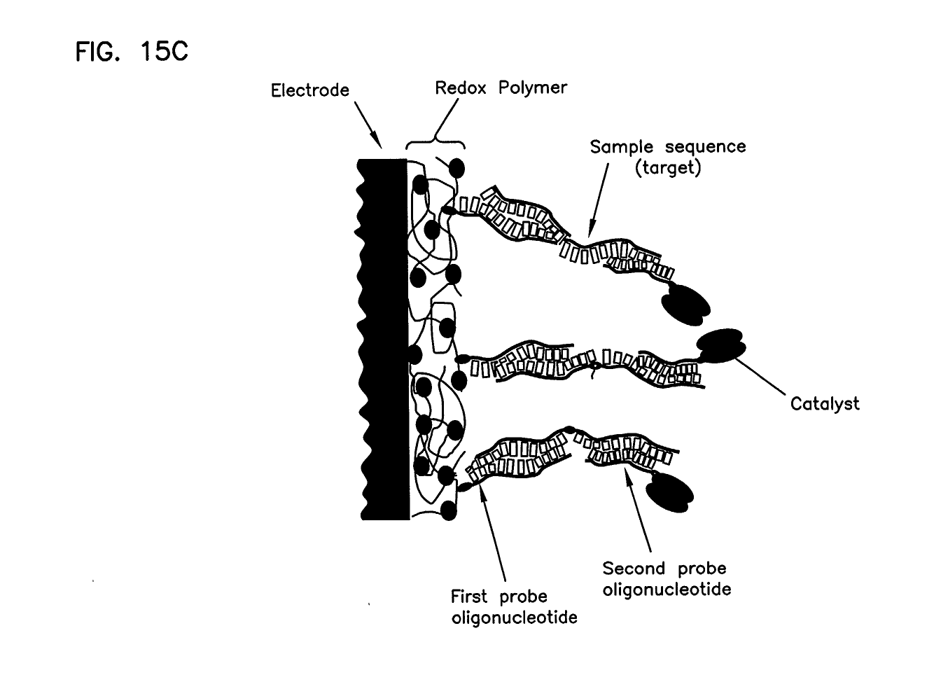

An array 100 of oligonucleotide labeled-sensors 101 is illustrated in FIG. 1. Each of the sensors 101 includes a working electrode 102, a redox polymer 104 deposited on the working electrode 102, and sensor oligonucleotides 106 coupled to the redox polymer 104, as shown in FIG. 2 and FIG. 15A. The sensor oligonucleotides 106 of each particular working sensor 101 may have the same sequence, but the sequence of the sensor oligonucleotides 106 preferably varies between at least two of the sensors 101, and, in general, the majority of sensors each contain a unique oligonucleotide sequence. Preferably, the array includes at least 4, 100, 1000, 10,000, or more sensors with each sensor oligonucleotide 106 having a specific sequence of nucleic acids. In alternative embodiments, the array includes fewer than 10 sensors, for example, in a discrete diagnostic device, as described below.

In an alternative embodiment, strips to identify one or a few pathogens are also envisaged. These can be, but need not be particularly small. Such devices would serve as an inexpensive and fast test to:

a) identify whether the infectious pathogen is a gram-positive or a gram-negative organism for subsequent appropriate antibiotic therapy;

b) identify specific type of infection: e.g. whether an infection of the throat is streptococcal; or

c) c) identify the location of an infection; e.g. whether the infection is localized or systemic (blood-borne).

As the majority of infectious diseases treated by physicians are E. coli in origin, a strip identifying and typing an infection as related to Staphylococcus aureus or Streptococcus would be particularly useful for determining subsequent therapy. An array of about 100 sensors would be sufficient to identify nearly all viral, fungal and bacterial pathogens treated currently by physicians.

In general, the array 100 is made by forming the working electrodes 102 on a substrate 114. A redox polymer 104 is then electrophoretically deposited on each working electrode 102. The redox polymer 104 typically includes a polymer and a redox species, such as a transition metal complex, coupled to the polymer. The polymer also typically includes reactive binding sites for the sensor oligonucleotide.

The sensor oligonucleotide is preferably treated to form a reactive functional group that reacts with the reactive binding sites of the polymer to couple the sensor oligonucleotide to the redox polymer. The sensor oligonucleotides are selectively deposited on a given electrode by “reactive electrophoresis”, a process that includes applying to the electrode a potential sufficient to cause migration of the ionic oligonucleotide to the surface of the electrode or its proximity. The sensor oligonucleotides are then coupled to the redox polymer via the reactive binding sites on the polymer of the redox polymer. One or more of the migrating oligonucleotide, the electrode surface, the chemical surface modifier (if present), and/or the polymer on the electrode may be chemically activated to cause the binding reaction.

When using the array of the invention in the probing process, target oligonucleotide 108 (e.g., a DNA or RNA segment) is brought into contact with the array under specific hybridization conditions. If the target oligonucleotide 108 contains a sequence that is complementary to any of the sensor oligonucleotides 106, the sensor and target oligonucleotides 106 and 108, respectively, hybridize, as shown in FIG. 3 and FIG. 15A.

In one embodiment, each of the target oligonucleotides 108 is coupled to a catalyst 110, that catalyzes an electrochemical reaction of a detection compound 112. Thus, when the detection compound 112 is brought into contact with the array 100 and a potential is applied to the working electrodes 102, an electrical signal is generated at the working electrode or working electrodes where the oligonucleotides 106, 108 have hybridized. This signal is generated through the electrochemical reaction of the detection compound 112.

In an alternative embodiment of the invention shown in FIG. 15C, a target nucleic acid sequence 107 hybridizes to both sensor oligonucleotide 106 and to a second, catalyst-labeled oligonucleotide probe 109. Hybridization of the target sequence 107 to both the first immobilized sensor sequence 106 and the second labeled sequence 109 results in the generation of an electrical signal at the working electrode 102, as described above.

The Array

The array 100 of sensors 101 is formed on a substrate 114. The material used to form the substrate 114 is typically insulating or semiconducting. Suitable materials, include, for example, silicon, fused silicon dioxide, silicate glass, alumina, aluminosilicate ceramic, epoxy, an epoxy composite such as glass fiber reinforced epoxy, polyester, polyimide, polyamide, or polycarbonate.

The array 100 of sensors 101 may be a regular array or an irregular array, and includes a plurality of sensors. In one embodiment, the array 100 includes sensors arranged in rows and columns. The array may include a plurality of sensors, for example, 4 or more sensors, preferably, 10 or more sensors, more preferably, 100 or more sensors, even more preferably, 1000 or more sensors, and most preferably, 10,000 or more sensors. The size of the array and the density of sensors on the array will vary with the desired use for the array. For example, as described above, strips to identify one or a few pathogens are envisaged, which can be, but need not be particularly small. Such an array would require fewer (about 100) sensors to identify nearly all viral, fungal and bacterial pathogens treated by physicians. Alternatively, a discrete diagnostic device may utilize an array of about four sensors to test for the presence of a nucleic acid sequence correlated with a metabolic disorder. In contrast, an array of about 100 sensors can be used to screen a sample for a variety of pathologic conditions. Alternatively, an array of about 10,000 or more individual sensors can be used to screen for nucleic acid mutations. In general, the spacing between the sensors exceeds the diameter of the sensors. Preferably, the spacings are at least twice the diameter and, more preferably, at least ten times the diameter.

For some applications, including diagnostic analysis of a specific nucleic acid, for example, to identify a pathogen, the number of sensors on the array may be small, for example, two or more. For diagnosis of specific nucleic acid variation or mutation, the number of sensors on the array might be about 10 or more, or even 100 or more. For identification of bioconjugation reactions of oligonucleotides or peptides, the array may include 10,000 or more individual sensors. For identification of a particular microbe in a patient sample, an array containing approximately 100 known diagnostic oligonucleotides can be provided for simultaneous analysis of a single sample. The size of each sensor within the array can also vary with the desired use, and can be, but need not be, miniaturized, for example, about 1-10 micrometers in diameter.

The Working electrodes

The working electrodes 102 are typically thin films of conductive material disposed on an insulating substrate 114, as shown in FIG. 1. A variety of conductive materials can be used to form the working electrodes 102. Suitable materials include, for example, metals, carbon, conductive polymers, and metallic compounds. Examples of these materials include gold, silver, copper, palladium, tantalum, tungsten, aluminum, graphite, titanium nitride, and ruthenium dioxide. The preferred materials do not corrode rapidly in aerated water when a potential of 0.2 volts positive of the potential of the saturated calomel electrode (SCE) is applied. The corrosion current density is preferably less than 10³ A cm⁻², and more preferably less than 10⁶ A cm⁻².

Thin films of these materials can be formed by a variety of methods including, for example, sputtering, reactive sputtering, physical vapor deposition, plasma deposition, chemical vapor deposition, printing, and other coating methods. Discrete conductive elements may be deposited to form each of the working electrodes, for example, using a patterned mask. Alternatively, a continuous conductive film may be applied to the substrate and then the working electrodes can be patterned from the film.

Patterning techniques for thin films of metal and other materials are well known in the semiconductor art and include photolithographic techniques. An exemplary technique includes depositing the thin film of conductive material and then depositing a layer of a photoresist over the thin film. Typical photoresists are chemicals, often organic compounds, that are altered by exposure to light of a particular wavelength or range of wavelengths. Exposure to light makes the photoresist either more or less susceptible to removal by chemical agents. After the layer of photoresist is applied, the photoresist is exposed to light, or other electromagnetic radiation, through a mask. Alternatively, the photoresist is patterned under a beam of charged particles, such as electrons. The mask may be a positive or negative mask depending on the nature of the photoresist. The mask includes the desired pattern of working electrodes. Once exposed, the portions of the photoresist and the thin film between the working electrodes is selectively removed using, for example, standard etching techniques (dry or wet), to leave the isolated working electrodes of the array.

The working electrodes can have a variety of shapes, including, for example, square, rectangular, circular, ovoid, and the like. The working electrodes can be very small, for example, having a dimension (e.g., length, width, or diameter) of 50 μm or less, preferably, 10 μm or less, and more preferably, 5 μm or less. In some embodiments, the working electrodes are three dimensional structures. Typically, the miniaturized working electrodes of the invention have a surface area of 1×10⁻⁴ cm² or less, preferably, 1×10⁻⁵ cm² or less, more preferably, 1×10⁻⁶ cm² or less, and, most preferably, 1×10⁻⁷ cm² or less. Typically, the density of the small sensors on a substrate is about 1000 sensors/cm² or more and, preferably, 10,000 sensors/cm² or more.

The electrodes 102 are connected to contact pads 120 for the application of a potential, for example, by vias (not shown) through the substrate; by conducting lines 122 (also known as “runners”) formed on the substrate 114 with the working electrodes 102 (as shown in FIG. 1); and/or by conducting lines formed on a silicon substrate then covered by a dielectric material upon which the working electrodes are formed with vias through the dielectric material to the conducting lines. If conducting lines (or “runners”) are formed on the substrate, then these conducting lines are insulated from exposure to the oligonucleotides by an inorganic or organic overlayer.

When a semiconducting or photoconducting material is used, the electrode can be illuminated with photons of energies greater than the band gap, to produce the desired potential on a particular site or element, or to transiently convert a particular microzone from being an insulator to being a conductor, thus electrically connecting the zone while it is illuminated.

The counter and reference electrodes may be present in the electrolytic solution off the surface of the substrate containing the array of working electrodes. Alternatively, the counter and reference electrodes may be part of the substrate or “chip” containing the array, for example, located on the same or a different surface as the working electrodes. It is not necessary for each working electrode to have a dedicated counter electrode or reference electrode. The same counter or reference electrode can serve multiple, or even all, electrodes of the array.

Preferably the reference electrode is one that does not leach ions and maintains a constant potential. The reference electrode can be, for example, a silver wire or structure, in contact with the electrolytic solution. The surface of the silver wire or structure is partially oxidized to produce Ag⁺Cl⁻ chemically, electrochemically, or otherwise.

The Flow Channel Array

The multisensor array may have a flow-channel through which the solution to be assayed is passed over multiple sensors. The target DNA or RNA is then captured from the sample solution passing over the sensor. The flow channel may be formed from a plastic, silicon, or ceramic material, or any other suitable material. Preferably, in order to reduce the volume of the sample solution required for probing, the width of the channel will be 200 μm or less. Because the channel is narrow, passage of the liquid through the channel may involve pumping techniques such as the use of a pressurized fluid including but not limited to air; or the application of a potential between two or more electrodes to drive the solution through the narrow channel.

The Redox Polymer

The redox polymer 104 is deposited on the working electrodes 102. Typically, redox polymer 104 is not deposited on the substrate 114 between the working electrodes 102, thus maintaining the electrical isolation between working electrodes 102. Redox polymers generally provide for adequate transport of electrons to and from the electrode if the redox polymer includes active redox functional groups that are mobile. For example, one type of redox polymer is a redox hydrogel which typically contains a large amount of water. Water soluble reactants and products often permeate through the redox hydrogel nearly as fast as they diffuse through water. Electron conduction in the redox hydrogel is through electron exchange between polymer segments that are mobile after the hydrogel is hydrated.

In general, the redox polymer includes electroreducible and electrooxidizable ions, functionalities, species, or molecules having redox potentials that are a few hundred millivolts above or below the redox potential of the standard calomel electrode (SCE). The preferred redox polymers include a redox species bound to a polymer that can in turn be immobilized on the working electrode. The polymer also includes binding sites for the oligonucleotide. Preferably, the redox polymers are not more reducing than about −400 mV and not more oxidizing than about 800 mV versus SCE, and most preferably not more reducing than about −150 mV and not more oxidizing than about +400 mV versus SCE at neutral pH. The most preferred redox polymers have osmium, ruthenium, or cobalt redox centers and a redox potential ranging from about −150 mV to about +400 mV versus SCE.

In general, redox polymers suitable for use in the invention have structures or charges that prevent or substantially reduce the diffusional loss of the redox species during the period of time in which the sample is being analyzed. The bond between the redox species and the polymer may be covalent, coordinative, or ionic. Useful redox polymers and methods for producing them are described in U.S. Pat. Nos. 5,264,104; 5,356,786; 5,262,035; 5,320,725; and 5,665,222, incorporated herein by reference. Although any organic or organometallic redox species can be bound to a polymer and used as a redox polymer, the preferred redox species is a transition metal compound or complex. The preferred transition metal compounds or complexes include osmium, ruthenium, iron, and cobalt compounds or complexes. The most preferred are osmium compounds and complexes.

One type of polymeric redox polymer contains a redox species covalently bound in a polymeric composition. An example of this type of mediator is poly(vinylferrocene).

Another type of redox polymer contains an ionically-bound redox species. Typically, this type of mediator includes a charged polymer coupled to an oppositely charged redox species. Examples of this type of redox polymer include a negatively charged polymer such as Nafion® (DuPont) coupled to a positively charged redox species containing one or more of osmium or ruthenium polypyridyl cations. Another example of a redox polymer comprises an ionically-bound positively charged polymer such as quaternized poly(4-vinyl pyridine) or poly(1-vinyl imidazole) and a negatively charged redox species such as ferricyanide or ferrocyanide. Thus, when the bonding is ionic the redox polymer may consist of a highly charged redox species that itself may be polymeric, bound within an oppositely charged redox polymer.

In another embodiment of the invention, suitable redox polymers include a redox species coordinatively bound to a polymer. For example, the mediator may be formed by coordination of an osmium or cobalt 2,2′-bipyridyl complex to poly(1-vinyl imidazole) or poly(4-vinyl pyridine).

The preferred redox species are transition metal complexes, most preferably of osmium, ruthenium, or cobalt, comprising one or more ligands, each ligand having a nitrogen-containing heterocycle such as 2,2′-bipyridine, 1,10-phenanthroline, 2,2′,2″-terpyridine, or derivatives thereof. More preferred redox species include osmium cations complexed with two ligands, each ligand containing 2,2′-bipyridine, 1,10-phenanthroline, or derivatives thereof, the two ligands not necessarily being the same. In the preferred complexes of osmium, ruthenium, or cobalt, the ion has six coordination sites, of which three or more are nitrogen-occupied, and the number of ligands ranges from 1 to 3. In the most preferred complexes, five of the coordination sites are nitrogen-occupied, and the number of ligands ranges from 2 to 3.

The preferred redox species exchanges electrons rapidly between each other and the working electrode so that the complexes can be rapidly oxidized and reduced. The preferred redox species are coordinatively or covalently bound to the polymer. Preferred polymers for coordinative-bonding have nitrogen-containing heterocycles, such as pyridine, imidazole, or derivatives thereof, binding as ligands to the redox species.

Preferred polymers for complexation with redox species, such as the osmium transition metal complexes, described above, include polymers and copolymers of poly(1-vinyl imidazole) (referred to as “PVI”), poly(4-vinyl pyridine) (referred to as “PVP”), and pyridinium-modified poly(acrylic acid). Suitable copolymer substituents of poly(1-vinyl imidazole) include acrylonitrile, acrylamide, acrylhydrazide, and substituted or quaternized N-vinyl imidazole. The osmium transition metal complexes coordinatively bind with the imidazole and pyridine groups of the polymer. Typically, the ratio of osmium transition metal complexes to imidazole and/or pyridine groups ranges from 1:10 to 1:1, preferably from 1:2 to 1:1, and more preferably from 3:4 to 1:1. Also, the preferred ratio of the number of complexed transition metal atoms and polymerized vinyl functions ranges from about 1:2 to about 1:30, and more preferably from about 1:5 to about 1:20.

Examples of other redox species include quinones and species that in their oxidized state have quinoid structures, such as Nile blue and indophenol. The preferred quinones and quinoids do not have hydrogen atoms in their six-membered rings.

The polymer also includes binding sites for the sensor oligonucleotides. In one embodiment, the sensor oligonucleotides are bound to the polymer by carbodiimide coupling to hydrazide functions on the polymer. The hydrazide functions may be provided by a variety of methods.

For example, in one embodiment, the polymer is a copolymer of PVI or PVP with polyacrylarnide (referred to as “PAA”). The osmium transition complex is coupled to the imidazole or pyridine groups of the PVI or PVP component, respectively. To form binding sites for the sensor oligonucleotides, a portion of the amide groups of the acrylamide is converted to hydrazide groups by known processes. Typically, at least 5% of the amide groups are converted, preferably, at least 10% of the groups are converted, and more preferably, at least 20% of the groups are converted. The ratio of PVI or PVP to PAA is typically 5:1 to 1:15, preferably, 2:1 to 1:12, and, more preferably, 1:1 to 1:10.

In another embodiment, the polymer is a cross-linked combination of the copolymer of PVI or PVP with PAA and with a hydrazide-modified PAA polymer (referred to as “PAH”). The ratio of hydrazide-modified amide groups to unmodified amide groups of the PAH polymer is typically 1:1 to 1:20, and preferably 1:2 to 1:10. The ratio of PAH to the copolymer of PVI or PVP with PAA is typically 1:5 to 2:1, preferably, 1:3 to 1:1. The two polymers can be cross-linked using the hydrazine modified groups of each polymer, as described below.

In yet another embodiment, the polymer is a modified poly(acrylic acid). A portion of the carboxylic acid functionalities of the poly(acrylic acid) are treated with a pyridine or imidazole reactive agent to attach pyridine or imidazole groups to the polymer. Such groups include, for example, 4-(aminoalkyl)-pyridine, such as 4-(2-aminoethyl)-pyridine, that can be attached using carbodiimide coupling. The pyridine and imidazole groups can then be used for coupling the osmium transition metal complexes. Typically, at least 2%, preferably, at least 5%, and, more preferably, at least 10%, of the carboxylic acid functionalities are treated with the pyridine or imidazole reactive agent. At least a portion of the remaining carboxylic acid groups are functionalized to hydrazide groups for coupling to the oligonucleotide. Typically, at least 2%, preferably, at least 5%, and, more preferably, at least 10%, of the carboxylic acid groups are functionalized to hydrazide groups.

A variety of methods may be used to immobilize a redox polymer on an electrode surface. One method is adsorptive irnmobilization. This method is particularly useful for redox polymers with relatively high molecular weights, for example, greater than about 10⁴ daltons, preferably greater than 10⁵ daltons, and most preferably greater than 10⁶ daltons. The molecular weight of a polymer may be increased, for example, by cross-linking with a di- or polyfunctional cross-linking agent, such as those listed in the Pierce catalog, 1994, pages T155-T167. Examples of functions of cross-linking agents useful in the invention include epoxy, aldehyde, N-hydroxysuccinimide, halogen, imidate, thiol, and quinone functions. Examples of crosslinkers include difunctional poly(ethylene glycol) and cyanuric chloride. Specific examples of useful crosslinkers include poly(ethylene glycol) diglycidyl ether (PEGDGE) of 400 or 600 daltons. Other cross-linking agents may also be used. In some embodiments, an additional cross-linking agent is not required.

In another embodiment, the redox polymer is immobilized by the functionalization of the electrode surface and then the chemical bonding, often covalently, of the redox polymer to the functional groups on the electrode surface. One example of this type of immobilization begins with a poly(4-vinylpyridine). The polymer's pyridine rings are, in part, complexed with a reducible/oxidizable species, such as [Os(bpy)₂Cl]^{+/2+} where bpy is 2,2′-bipyridine. Part of the pyridine rings are quaternized by reaction with 2-bromoethylamine. The polymer is then crosslinked, using, for example, using a diepoxide, such as poly(ethylene glycol) diglycidyl ether.

Carbon surfaces can be modified for attachment of a redox species or polymer, for example, by electroreduction of a diazonium salt. As an illustration, reduction of a diazonium salt formed upon diazotization of p-aminobenzoic acid modifies a carbon surface with phenylcarboxylic acid functional groups. These functional groups can then be activated by a carbodiimide, such as 1-ethyl-3-(3-dimethylaminopropyl)-carbodiimide hydrochloride. The activated functional groups are then bound with a amine-functionalized redox couple, such as, for example, the quaternized osmium-containing redox polymers described above or 2-aminoethylferrocene, to form the redox couple.

Similarly, gold and other metal surfaces can be functionalized by an amine, such as cystamine. A redox couple such as [Os(bpy)₂(pyridine-4-carboxylate)Cl]^{0/+} is activated by 1-ethyl-3-(3-dimethylaminopropyl)-carbodiimide hydrochloride to form a reactive O-acylisourea which reacts with the gold-bound amine to form an amide.

The Sensor Oligonucleotides

Sensor oligonucleotides 106 are nucleic acid sequences immobilized onto the working electrodes and usefull as hybridization probes for hybridizing to target nucleic acid sequences. Sensor oligonucleotides include DNA or RNA sequences, also include those DNA or RNA sequences having attached peptides, which oligonucleotides are useful for specific diagnostic or immobilization purposes and include peptide nucleic acids (PNA; also known as protein nucleic acids). PNA are useful as hybridization probes because they bind single-stranded DNA or RNA more strongly due to their absence of negatively charged phosphate functions which reduces electrostatic repulsion between the segments of the hybrids. This stronger bonding results in higher melting temperature of the hybrids formed. Due to the stronger bonding, shorter segments are required for recognition. Typically, 8-base PNAs can be effectively used as a probe. As described for the DNA and RNA probes, PNA probes can be coupled to a label, for example, a catalyst capable of catalyzing an electrochemical reaction of a detection compound. As with DNA or RNA probes, PNA probes can be coupled to the redox polymer of specific electrodes.

The sensor oligonucleotides may consist of conventional nucleotides, synthetic nucleotides, peptide-nucleotides, and the like. The sensor oligonucleotide typically includes a sequence of nucleotides useful for hybridization, and, may be, for example, about 5 to about 300 nucleotides in length, and preferably about 8 to about 20 nucleotides. Each working electrode is formed using a selected sensor oligonucleotide, that may contain the same or different sequences as the sensor oligonucleotides of other working electrodes. In general, each working electrode contains a unique sensor oligonucleotide sequence, subject to a desired redundancy in the array. Thus, for example, at least two of the working electrodes 102, preferably at least 4 of the working electrodes, more preferably, at least 10 of the working electrodes, even more preferably, at least 100 of the working electrodes, and most preferably at least 1000 of the working electrodes in a sensor array have sensor oligonucleotides with differing nucleotide sequences.

The specific nucleotide sequence of the sensor oligonucleotide is selected for the desired application. For example, an array useful for the detection of gene mutations associated with risk for a particular disease, e.g., cancer, will contain one or more oligonucleotide sensor sequences designed to specifically hybridize and identify the known gene mutations. A diagnostic array for screening a blood sample for the detection of specific pathogenic microorganisms will contain one or more oligonucleotide sensor sequences designed to specifically hybridize and identify the pathogen. Adaption of the inventive arrays to achieve detection and discrimination of nucleic acid sequences for a wide variety of specific applications will be readily understood by one of skill in the field of nucleic acid sequencing.

Binding Sensor Oligonucleotides to the Electrode

The sensor oligonucleotides are generated using known techniques. The sensor oligonucleotides are prepared for coupling to the redox polymer by addition of a reactive group, preferably, to one end of the oligonucleotide. The particular reactive group that is used typically depends on the functionality of the reactive site on the redox polymer and on the possibilities of side reactions.

In one embodiment, the reactive sites on the redox polymer are hydrazides, as described above. In this embodiment, a suitable reactant is the 5′-phosphate ester of the oligonucleotide. The oligonucleotide is dissolved in a solvent, such as water, and combined with a 0.1 M 1-methylimidazole buffer. The oligonucleotide solution is then combined with a carbodiimide to activate the phosphate ester. An exemplary carbodiimide is 1-ethyl-3-(3-dimethylaminopropyl)-carbodiimide hydrochloride (EDC). Alternatively, oxidation at the 3′ end with periodate produces the aldehyde group that reacts with the hydrazine moieties. Once the phosphate group is activated, the oligonucleotide solution is brought into contact with the electrodes having hydrazide active binding sites.

To selectively bind the oligonucleotide onto a particular electrode or electrodes, a potential is provided across the electrode or electrodes to attract the oligonucleotide, causing its migration to the electrode, a process known as electrophoresis. This process is preferably carried out at low ionic strength, such as less than 0.1 M NaCl, more preferably less than 0.001 molar NaCl, and most preferably in the absence of any added salt and using the purified reactants.

In the electrophoretic process, the attraction arises, usually because the conventional oligonucleotide includes anionic functionalities, and because a potential, causing the electrode to have a positive charge, is applied. In some applications, the oligonucleotides to be immobilized contain an attached peptide sequence. Peptides can include both cations and anions. For specific embodiments, the pH of the solution, the potential applied, and the isoelectric point of the oligonucleotide solution may be modified to modulate the electrophoretic or ionic migration of the specific oligonucleotide to the desired electrode. Because the oligonucleotides are preferentially attracted to the electrode or electrodes to which an appropriate potential is applied, the oligonucleotides are preferentially coupled to the redox polymer deposited on those electrodes.

In some embodiments, a potential is applied to specific electrodes to repel the oligonucleotides and prevent their deposition. Natural oligonucleotides are polyanions at neutral pH and are attracted to electrodes to which a positive potential is applied and repelled by electrodes to which a negative potential is applied. Knowing the specific ionic character of the molecule to be attached an appropriate potential can be applied at each of the electrodes in the array to selectively attract or repel the molecule to each electrode of the array, as desired.

The Probe Oligonucleotides

Probe oligonucleotides of the present invention include labeled target nucleic acid sequences 108 or second labeled hybridization probes 109. The labeled oligonucleotides are prepared by coupling a label, for example, a catalyst to the oligonucleotide. The catalyst catalyzes an electrochemical reaction of a detection compound. Preferably, the electrochemical reaction of the detection compound is the electrooxidation or electroreduction of the detection compound. This electrochemical reaction transduces a current at the electrode via the redox polymer and catalyst. In the embodiment shown in FIG. 15A, because the probe oligonucleotide preferentially binds to a sensor oligonucleotide having a complementary sequence of nucleotides, a current is generated at those sensors having the appropriate sensor oligonucleotides. By observing those electrode(s) that have a current above a threshold value, a determination of at least a portion of the sequence of the probe oligonucleotide can be made. A current at more than one electrode may occur if the probe oligonucleotide has portions that are complementary to more than one of the sensor oligonucleotides.

In the embodiment shown in FIG. 15C, hybridization of a target nucleic acid sequence to both a sensor oligonuclotide and a labeled probe oligonucleotide electrically couples the catalyst attached to the probe oligonucleotide to the working electrode. Those electrodes having a current above a threshold value demonstrate hybridization events.

Binding of the Sensor Oligonucleotide to the Electrode

It is preferred that the binding of the first electrode-modifying oligonucleotide to the redox polymer on the electrode be through covalent bonds, rather than electrostatic bonds. In the most preferred embodiment, the redox polymer contains functional groups that react to form covalent bonds with the oligonucleotide. When a polyanionic oligonucleotide is used to modify the redox polymer, the charge on the redox polymer at neutral pH and at low ionic strength (0-0.1 M NaCl) can be negative, neutral, or very slightly positive, with fewer than one positive charge per 5-mer, preferably with fewer than one positive charge per 10-mer, and most preferably fewer than one positive charge per 20-mer.

When a polyanionic oligonucleotide is used as a probe, it is also preferred that there be no strong electrostatic interaction between the oligonucleotide-modified redox polymer and the probe, including the complementary oligonucleotide labeled with an enzyme catalyst. Because probing, where the sensor oligonucleotide and probe oligonucleotide bind, or hybridize, can be carried out at high ionic strength, for example greater than about 0.5 M NaCl, the extent of electrostatic interaction can be reduced by adding a salt and sufficiently raising the ionic strength. It is usually sufficient to add NaCl in an amount required to bring its concentration to about 0.5 M and preferably to about 1 M. The extent of electrostatic interaction of the enzyme-labeled probe oligonucleotide with the oligosaccharide-modified redox polymer can also be reduced by using an enzyme label that is negatively charged, is neutral, or is only slightly positive at pH 7. The preferred enzyme labels have isoelectric points (pI) of about 8 or less, more preferred are those with a pI of about 6.5 or less, and most preferred having a pI of about 5 or less (the isoelectric points being expressed in pH units).

Blocking buffers, detergents such as TWEEN, and other known methods may be used to reduce the non-specific binding of the probe oligonucleotide.

A peptide oligonucleotide (e.g., hybrid synthetic molecule containing both nucleic acids and amino acids) is not necessarily negatively charged at pH 7. When a peptide-oligonucleotide is used as the sensor oligonucleotide, the redox polymer can have a substantial positive charge and/or the probe oligonucleotide may be labeled with an enzyme that need not have a particularly low isoelectric point, as required with use of conventional oligonucleotides(containing no amino acids). When one member of the hybridization pair (sensor oligonucleotide and probe oligonucleotide) is a natural oligonucleotide and the other is a protein- or peptide-oligonucleotide (combination of an amino acid sequence and a nucleic acid sequence), weakly charged redox polymers and enzyme labels are preferred. The charge of the redox polymers and enzyme labels can be adjusted by controlling the pH of the solution from which the oligonucleotides are electrophoretically deposited to react with the redox polymer, as well as the pH of the solution in which the enzyme labeled probe oligonucleotide is allowed to hybridize with the sensor oligonucleotide.

After hybridization, the probe oligonucleotides can be removed from the sensor oligonucleotides by a variety of methods. Exemplary methods include the use of heat or chemicals that denature (melt) the hybridized bonds between the oligonucleotides.

The Catalyst

A variety of catalysts can be used in the invention. Exemplary catalysts are enzymes that catalyze an electrochemical reaction of a detection compound. A variety of enzymes are useful including, for example, peroxidases for use with hydrogen peroxide, glucose oxidase and glucose dehydrogenase for use with glucose, and lactate oxidase and lactate dehydrogenase for use with lactate. Preferably, thermostable enzymes (enzymes capable of operation for at least 1 hour at 37° C.) are used. Thermostable enzymes are capable of operation for at least 1 hour at 37° C. As used herein, the term “capable of operation” means that the enzymes loses less than 30%, more preferably less than 10% of its activity following heating. When similar sequences need to be sorted, as is the case, for example, when mutants are to be detected or when cancer or a genetic disease is to be diagnosed, then it is preferred to use enzymes with a higher thermostability such that when operating at about 5° C. below the melting temperature of the hybrid formed in the recognition process, the enzyme retains, for >1 hour, about 70% or more of its enzymatic activity. Soybean peroxidase is one example of a useful thermostable

The catalyst may also be an enzyme accelerating the hydrolysis of a precursor compound to an electrooxidizable or electroreducible compound. An example of such a catalyst is alkaline phosphatase, an enzyme accelerating the hydrolysis of the phosphate ester derivative of p-aminophenol to form electrooxidizable p-aminophenol.

The Coupling of the Catalyst to the Probe Oligonucleotides

The probe oligonucleotide is coupled to the catalyst by a variety of methods. In particular, the probe oligonucleotide can be prepared with reactive functionalities to reactively couple the probe with one or more functionalities on the catalyst.

In one embodiment, the 5′-phosphate ester of the probe oligonucleotide is activated in a solution containing 0.1 mM 1-methylimidazole buffer and EDC and reacted with hydrazine monohydrate to provide a monohydrazide. The enzyme is treated to generate aldehyde functional units, for example, by treatment with sodium periodate. The treated enzyme and hydrazide-functional oligonucleotide are reacted to form Schiff's bases (also known as hydrazones) between the aldehydes of the enzyme and the hydrazides of the oligonucleotides. These Schiffs bases are reduced using a reducing agent such as sodium borohydride to provide the catalyst-labeled oligonucleotide.

The Detection Compound

The detection compound is usually a compound (e.g., a substrate) that is reduced or oxidized in the presence of the labeling catalyst or enzyme, for example, hydrogen peroxide when the enzyme is a peroxidase. It can also be a compound that is easily electrooxidized or is easily electroreduced, and is formed in an enzyme-catalyzed hydrolysis reaction from an electrochemically less active precursor.

The Sample

In the method of the invention, a nucleic acid sample is analyzed for its ability to hybridize the oligonucleotide probes of the device/system. The sample nucleic acid may be DNA or RNA. Preferably, the sample is a body fluid or a tissue sample, for example, blood, urine, or feces. The sample is analyzed for the presence/absence of one or more particular nucleic acid sequences. When the sample nucleic acid is RNA (more preferable, for example, in the detection of pathogens and cancer), ribosomal RNA can be preferred because of its abundance and lower ratio of hydrolysis. RNA is produced in more abundance in pathogens and cells than is DNA (ratio can be 10⁴:1, respectively). In such a case, the length of the restriction endonuclease segment to be used is preferably, about 200-300 bases long. However, sequences of 50-1000 bases long can also be used.

The sample may be used directly. Preferably, the sample is treated to release the nucleic acids for hybridization. For example, a blood or fecal or urine sample (or other tissue sample) is treated to lyse cells. The released DNA and/or RNA is cleaved, for example, with endonuclease, to prepare nucleic acid fragments, preferably of about 250-300 base pairs. The nucleic acid fragments are denatured, e.g., by heat, to produce single-stranded sample, and used in the diagnostic assay system of the invention to analyze hybridization to specific probes. In the preferred method of the invention, the sample nucleic acid is not amplified, e.g., by PCR, but is directly used in the assay.

Operation of the Array

In the preferred mode of operation, the array is exposed to a solution or solutions containing a target nucleic acid sequence (DNA or RNA) which may be complementary to part or all of one or more of the sensor oligonucleotides immobilized on the array. The target sequence may contain an attached catalyst, preferably an enzyme label. Alternatively, the target sequence is unlabeled and a second labeled oligonucleotide sequence containing an attached catalyst (enzyme) label is added to the reaction. The same or a different solution to which the array is exposed contains the substrate of the catalyst (enzyme). After the sensor oligonucleotide is reacted under hybridization conditions with the target and/or enzyme-labeled probe oligonucleotide in solution, a potential is applied to the microelectrode.

The current associated with the occurrence of a reaction accelerated by the labeling enzyme is detected. The current may be the result of the electrooxidation or electroreduction of the substrate of the labeling enzyme or the result of the electrooxidation or the electroreduction of a product of the enzyme-catalyzed reactions, or, if multiple conversion steps are involved, the electrooxidation or the electroreduction of the end product of a sequence of reactions. For example, the current may be produced when the enzyme label is a peroxidase by the electroreduction of its substrate, hydrogen peroxide, to water. Hydrogen peroxide can be produced in situ from a stable precursor. For example, glucose, rather than hydrogen peroxide, can be added to the test solution. The hydrogen peroxide can be produced by adding to the solution glucose oxidase, known to catalyze the reaction of glucose and molecular oxygen to form gluconolactone and hydrogen peroxide. The added glucose oxidase may be immobilized, so as to improve its stability, for example in a hydrated silica gel, formed by the sol-gel process.

Any non-“wired” enzyme can be used to generate the detection compound. The detection compound is usually a substrate or co-substrate of the catalyst that is used for labeling the DNA, RNA, or PNA sequence applied in a recognition reaction. An example of an enzyme that catalyzes a reaction whereby a detection compound is generated is choline oxidase. Choline, unlike hydrogen peroxide, is a stable compound. The catalytic center(s) of choline oxidase do not exchange electrons with the redox polymer(s) on the electrode. The enzyme catalyzes the reaction of dissolved choline and dissolved oxygen, whereby hydrogen peroxide is generated. Thus, when choline oxidase is co-immobilized in the redox polymer on the electrode and choline is present in the solution, which contains dissolved air or oxygen, then the need to also add hydrogen peroxide is obviated.

Yet another example of generating a detection compound in the redox polymer is that of generating an air-oxidizable polyphenol, oxidized to a quinone, by incorporating a hydroxylase in the redox polymer. Quinones are co-substrates of e.g. flavoprotein enzymes, such as oxidases. Yet a third example is the incorporation of a hydrolase, whereby an air-oxidizable aminophenon, such as p-aminophenol is generated, for example, from the amide form by a proteolytic enzyme). Again the oxidized, quinoid-form of the product (p-aminophenol) is the substrate of oxidases and other redox enzymes.

Alternatively, the oligonucleotide-labeling enzyme may be glucose oxidase and the hybridization may result in the electrooxidation of glucose after hybridization.

Mismatch Discrimination

The melting temperature for hybridized complementary strands of an oligonucleotide can be calculated, for example, using the following equation:

Tₘ=81.5° C.−16.6(log₁₀[Na])+0.41(%G+C)−600/(length in bp)

For example, using an oligonucleotide of 18 base pairs, the melting temperature calculates to about 59° C. For a single base pair mismatch, the melting point is reduced by about 5° C., and for an oligo having four mismatches, the melting point is reduced by about 20° C. By analysis of the melting of an oligonucleotide to a target at different temperatures, discrimination between mismatches and complementary oligonucleotide sequences is achieved.

### EXAMPLES

The invention may be better understood by reference to the following Examples, that are not intended to limit the invention in any way.

### Example 1

### Preparation of Electrode with Redox Polymer

Materials

A 25-30 base single-stranded poly(deoxythymidine)-5′-phosphate (p(dT)₂₅₋₃₀) (cat. #27-7839-01), 12-18 base single-stranded poly(deoxyguanidine)-5′-phosphate (P(dG)₁₂₋₁₈) (cat. #27-7885-01) and 25-30 base single-stranded poly(deoxyadenosine)-5′-phosphate (p(dA)₂₅₋₃₀) (cat. #27-7986-01) were obtained from Pharmacia Biotech. Sodium periodate (cat. # 31,144-8), Tween 20 (cat. # 27,4343-8), and 1-(3-dimethlyaminopropyl)-3-ethylcarbodiimide hydrochloride (cat. #16,146-2) were purchased from Aldrich. Imidazole (cat. #I-20-2) and horseradish peroxidase (HRP) (cat. #P-8375) were purchased from Sigma. All measurements were carried out in a phosphate buffer at pH 7.0 and containing 0.4 M sodium chloride, unless otherwise stated.

10 μm diameter glassy carbon microelectrodes (cat. # EE017) were obtained from Cypress Systems (Lawrence, Kans.). The microelectrodes were polished with 1.0 and 0.3 μm alumina paste and sonicated in deionised water. The microelectrodes were stored in deionised water at all times before being used. Three millimeter diameter vitreous carbon macroelectrodes were similarly polished.

A computer controlled EG&G galvanostat Model 273, supported with EG&G M270 software was used in the electrophoretic deposition steps.

A polyacrylamide-poly(1-vinylimidazole) redox polymer with [Os[4,4′-dimethyl-2,2′ bipyridine]₂Cl]^{+/2+} redox centers was formed as described in de Lumley-Woodyear, et al., Anal. Chem. 43:1332-1338 (1995), incorporated by reference. The PAA-PVI-Os redox polymer was electrophoretically deposited from a 0.1 mg/mL de-ionized water solution onto 1-4 microelectrodes and the 3 mm diameter macroelectrode while the ensemble of these electrodes was shorted. By shorting the microelectrodes to the much larger macroelectrode, the area of which was easy to measure, it was possible to accurately determine the amount of material deposited per unit area in the electrophoretic process, through measuring the charge passed.

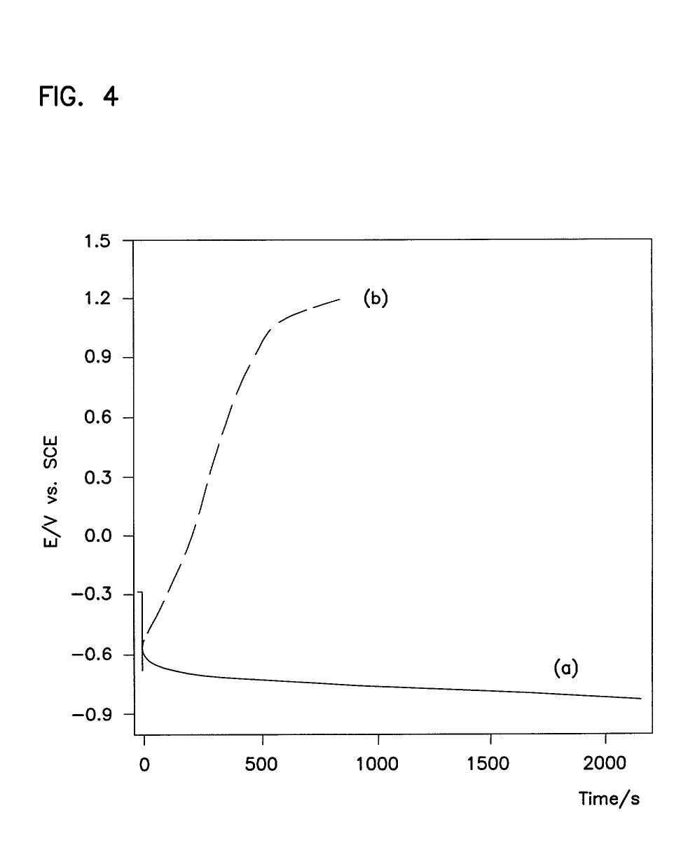

A current of +20 microamps was maintained for 30 minutes during which the integrated charge passed was 65 mC. FIG. 4 shows the evolution of the potential during this period. As shown in FIG. 4, the potential varied only slightly during the constant applied current deposition of the redox polymer on the microelectrodes, indicating that the layer deposited was not very resistive (see FIG. 4, curve a).

Films of reproducible thickness were deposited by shorting to the microelectrodes and to a 3 mm diameter vitreous carbon macroelectrode. The area of the macroelectrode defined, at constant applied current, the current density, and thereby the redox polymer film thickness. The amount of actually deposited electroactive polymer was determined coulometrically by integrating the reduction and oxidation waves of the cyclic voltammograms at 50 mVs⁻¹ scan rate. Of 30 attempts on three different electrodes (10 depositions on each electrode), 24 depositions were successful. The average integrated charge was 1.12×10⁻¹⁰ C, with a standard deviation of +/−0.09×10⁻¹⁰ C, suggesting that the amount of material deposited was reproducible within +/−8%. Reproducible films were similarly made of similar solutions and under similar conditions with the triepoxide crosslinker N,N-diglycidyl-4-glycidoxyaniline (5 μg/ml) added to the PAA-PUI-O_{S} redox polymer solution used for the electrophoretic deposition reaction.

Because of the very nature of the electrophoretic process, the deposition was confined to the conducting carbon surfaces. Examination by optical microscopy showed that the entire surface of the microelectrodes and the macroelectrode was uniformly coated with a shiny, purple redox polymer film, and that no polymer was deposited on the surrounding insulator.

### Example 2

### Preparation of Sensor Oligonucleotides and Coupling to Redox Polymer

The simple oligonucleotide sequence, pd(T)₂₅₋₃₀ was electrophoretically transported and covalently bound to the PAA-PVI-Os conducting redox polymer film on the electrode in a two step process. First, the terminal 5′-phosphate of the single-stranded oligonucleotide was activated by reacting it with EDC. Next, the active oligonucleotide was electrophoretically deposited on, and reacted with the redox polymer. Through this step, the single stranded oligonucleotide was covalently bound to NH₂ hydrazide functions of PAA-PVI-Os . The procedures used included the following steps:

The sensor oligonucleotide, 185 micrograms of pd(T)₂₅₋₃₀ was dissolved in 147 microliters of deionized water premixed with 27 microliters of 0.1 M imidazole. A volume of 50 microliters of 0.5 M EDC in deionized water was added and the activation reaction was allowed to proceed overnight at 4° C. A volume of 200 microliters of the activated pd(T)₂₅₋₃₀ solution was then diluted with deionized water to a volume of 2.5 milliliters, and this volume was used in the reactive electrophoretic deposition step.

For reactive electrophoretic deposition, a current of −10 microamps was applied to a single microelectrode for 900 seconds. The evolution of the potential during this step is shown in FIG. 4.

The reactive electrophoretic deposition of activated pd(T)₂₅₋₃₀ was self-limiting and thus reproducible. The potential increased steeply during the first 10 minutes, then leveled off, showing that upon deposition of a defined amount of pd(A)₂₅₋₃₀ the initially conductive redox polymer film became highly resistive (See FIG. 4, curve b). Thus, control of the current density through use of the auxiliary macroelectrode was not essential for reproducibility. In experiments on an individual microelectrode at an applied current of −0.3 nA, the potential was first stable for 10 minutes (−0.9 to −1.0V), then increased rapidly, showing that the amount of deposited material can be controlled simply through monitoring the potential and stopping the process when the end of the potential plateau is reached.

### Example 3

### Preparation of Peroxidase-labeled Oligonucleotide.

The oligonucleotides pd(A)₂₅₋₃₀ and pd(G)₁₈₋₂₀ were labeled with horseradish peroxidase (HRP) as described in deLumley-Woodyear, et.al., 1996 J. Am. Chem. Soc. 118:5504. In general, oligonucleotide monohydrazide termini were formed by EDC activation of 5′-phosphate functions and condensation with an excess of hydrazine. HRP-oligosaccharide alcohol functions were then oxidized with periodate to aldehydes. The aldehydes and the hydrazides were then condensed to form hydrazones.

Activities of the HRP-labeled oligonucleotides were derived from the measured protein concentrations and the rates of HRP-catalyzed hydrogen peroxide oxidation of the leucodye 2,2′-azino-bis(3-ethylbenzthiazoline-6-sulfuric acid) (ABTS) to the dye. Protein concentrations were measured using the BioRad Protein Assay Kit II. The rates of dye-formation were measured spectrophometrically, using a Hewlett Packard Diode Array UV/Vis, Model 8452A spectrophotometer. The procedure was to add 2.9 mL of 5 mM ABTS to 50 microliters of 0.1875 mg/mL of the HRP-labeled oligonucleotide solution, followed by 50 microliters of 60 mM hydrogen peroxide. The change in absorbance at 404 nm was recorded for 60 seconds.

A comparison of the activities of HRP, pd(A)₂₅₋₃₀-HRP, and pd(G)₁₈₋₂₀-HRP indicated that only 42% of the HRP activity was conserved after its attachment to either oligonucleotide, and that there was no measurable difference in the activities of the HRP labels of the two oligonucleotides. A fresh sample of HRP-labeled oligonucleotide was prepared for each set of experiments, although no loss of activity was observed after storage at 4° C. for one week.

### Example 4

### Sensing of Probe Oligonucleotides

Electrochemical measurements were performed in a Faraday cage using a water-jacketed, thermostatic, electrochemical cell with a pair of 10 micrometer diameter glassy carbon working microelectrodes, a silver/silver chloride Bioanalytical Systems reference electrode, and a platinum wire counter electrode. The current was monitored using a computer controlled CH Instruments Model 720 low noise bipotentiostat with CH Instruments software. The measurements were carried out in pH 7.0 phosphate buffer containing 0.4 M sodium chloride, unless otherwise stated.

The formation of nucleic acid hybrids was observed by measuring hydrogen peroxide electroreduction currents of a pair of the PAA-PVI-Os coated microelectrodes with pd(T)₂₅₋₃₀. The electrodes were immersed in hybridization solutions containing either 4×10⁻⁷ M complementary pd(A)₂₅₋₃₀-HRP or 4×10⁻⁷ M non-complementary pd(G)₁₈₋₂₀-HRP . The solutions were stirred and maintained at 4° C. In addition to the HRP-labeled oligonucleotide, the solutions contained 5×10⁻² M TRIS HCl; 1M NaCl; 0.2% TWEEN 20; 0.1 mM EDTA; and 4×10⁻² M of an unbound but active HRP residue from the labeling of the oligonucleotide. This unbound HRP did not contribute to the catalytic current (through non-specific absorption). After 20 minutes, the electrodes were removed from the hybridization solution, rinsed by dipping in buffer, transferred to a thermostated (25° C.) electrochemical cell containing 5 ml buffer and poised at 0.0V vs. Ag/AgCl. The electrodes were allowed to stabilize for 2 minutes, then 1 mM hydrogen peroxide was injected and the change in the catalytic electroreduction current was monitored.

Of the 17 hybridized microelectrodes completed and tested, 6 did not produce measurable currents because their redox polymer films were lost; one produced a current of 10 pA; one produced a current of 6 pA; and 9 produced similar currents of 20+/−2 pA.

Subsequently, for melting of the hybrids, the temperature of the cell was raised at a constant rate of 0.25° C. per minute while the change in current was monitored.

### Example 5

### Electrochemical Characterization of the System

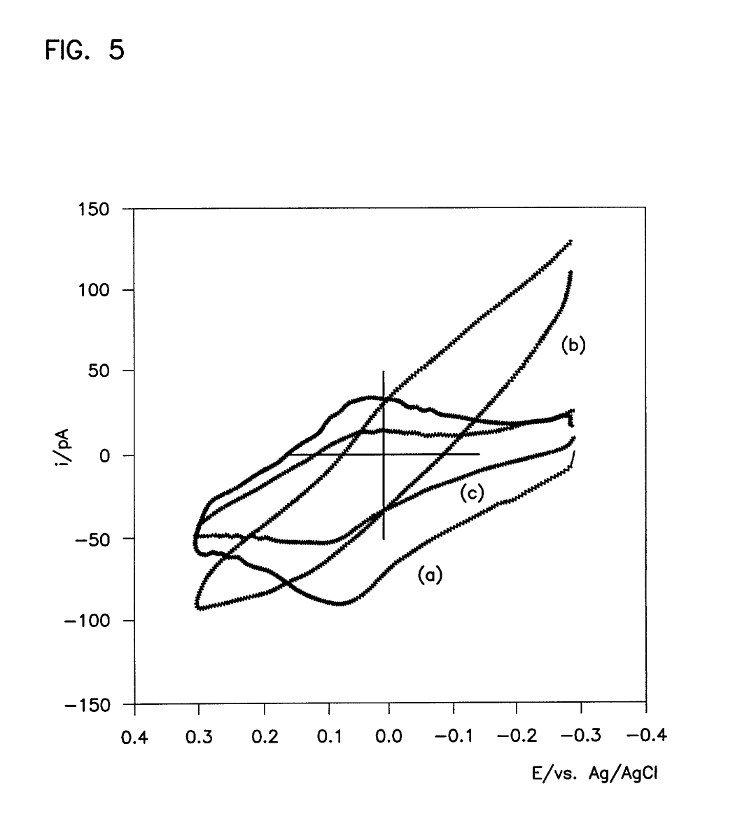

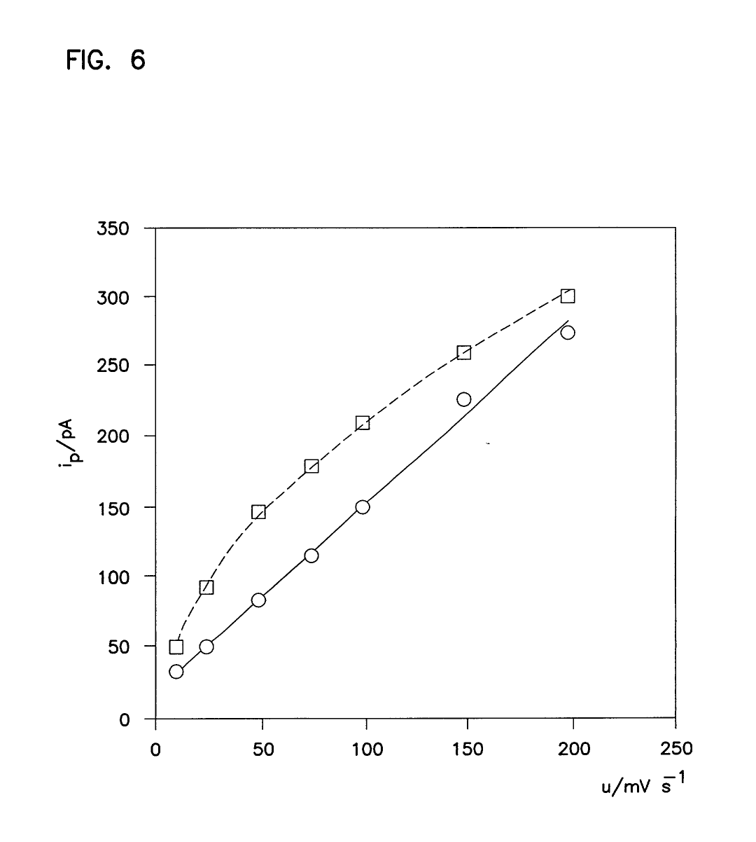

FIG. 5 shows the cyclic voltammograms for the microelectrodes produced as described for Example 4. The graphs show voltammograms for (a) the electrophoretically deposited PAA-PVI-Os redox polymer film; (b) the redox polymer film after covalently binding to it pd(T)₂₅₋₃₀ through reactive electrophoretic deposition of the latter; and (c) the bound pd(T)₂₅₋₃₀ containing film, after its hybridization with HRP-labeled pd(A)₂₅₋₃₀, and prior to adding hydrogen peroxide. The faradaic charge required for the electrooxidation of the fully reduced film, 1.12×10⁻¹⁻ C, corresponded to 1.16×10⁻¹⁵ moles of Os⁺². The redox potential of the polymer was +75 mV vs AglAgCl, as previously reported by Hacia et.al, 1996 Nature Genet. 14:441. The peak height of the reduction wave was proportional to the scan rate, indicative of an immobilized, surface bound redox polymer (FIG. 6). In contrast, the peak height of the oxidation wave was proportional to the square root of the scan rate, indicative of a substantial motion of segments of the redox polymer (Aoki et.al., 1995 J. Phys. Chem. 99:5102.) Such mixed behavior is expected of a film that is better hydrated and is thus less viscous when its redox centers are oxidized. At 50 mV/sec scan rate, the peak to peak separation was 45+/−5 mV, consistent with fewer mobile Os⁺²-loaded chain segments and more mobile Os⁺³-loaded chain segments.

Reactive electrophoretic binding of pd(T)₂₅₋₃₀ onto the redox polymer film drastically altered the voltammogram. Consistently with the above-described steep increase in the resistance of the films (FIG. 4), the resistance was so great that the reduction/oxidation waves were barely visible.

Following hybridization, but prior to adding hydrogen peroxide, the peaks of the electrooxidation and electroreduction waves were again better defined (FIG. 5C) and a decrease by 32+/−10% in the integrals of the reduction and oxidation waves, i.e., in the faradaic charge required for the electrooxidation and the electroreduction of the redox centers was observed.

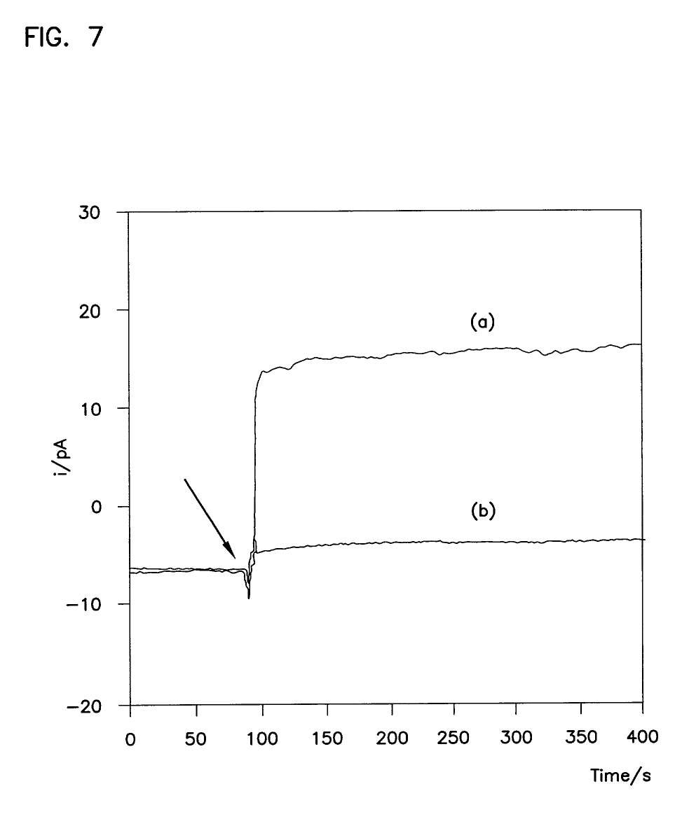

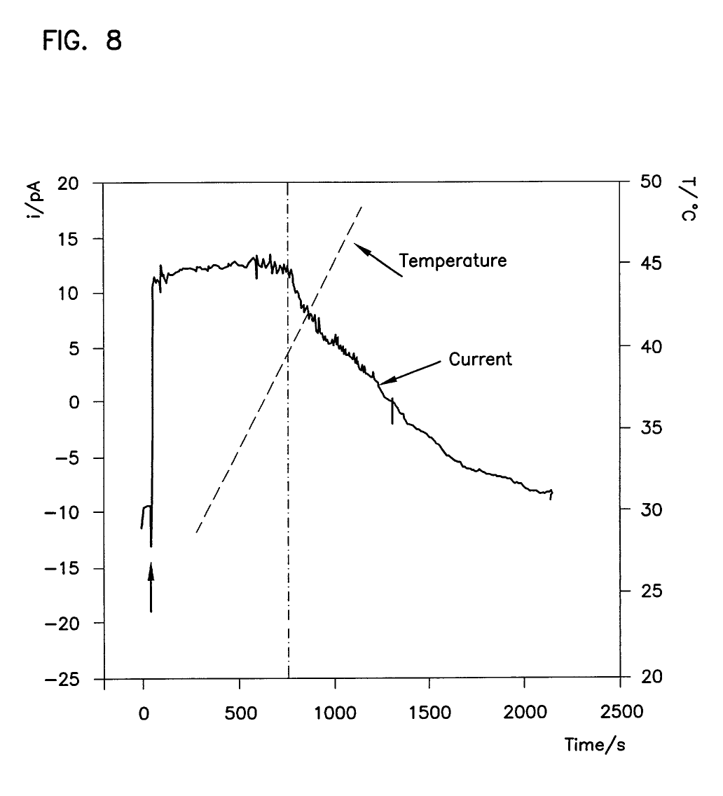

The change in the hydrogen peroxide electrocatalytic reduction current was measured simultaneously in pairs of microelectrodes after exposure of one microelectrode to the complementary pd(A)₂₅₋₃₀-HRP and exposure of the other electrode to the non-complementary pd(G)₁₈₋₂₀-HRP. The current of the microelectrode exposed to pd(A)₂₅₋₃₀-HRP increased upon injection of hydrogen peroxide by 20+/−2 pA (FIG. 8)), while the current of the electrode exposed to pd(G)₁₈₋₂₀-HRP increased only by 2.5+/−2.5 pA. The electrical noise in the measurements was 0.5 pA. When the pd(T)₂₅₋₃₀ was not activated with EDC prior to its electrophoretic deposition and was, therefore, not covalently bound to the redox polymer film, the hydrogen peroxide electroreduction current of the pd(A)₂₅₋₃₀-HRP exposed electrode was only 4+/−2 pA (FIG. 7), and that of the pd(G)₁₈₋₂₀-HRP exposed electrode was not measurable.

In a series of experiments on microelectrodes with the pd(T)₂₅₋₃₀ /pd(A)₂₅₋₃₀-HRP hybrid films, the temperature of the solution in the cell was linearly ramped from 25° C. to 49° C. at a rate of 0.25° C. per minute, and the hydrogen peroxide electroreduction current was monitored. As shown in FIG. 8, the current increased until the temperature reached 40° C., then decreased by 30% when the temperature was further raised by a single degree to 41° C.

### Example 6

### Electrochemical Mismatch Identification

Mismatch of an oligonucleotide was rapidly, efficiently, and specifically detected amperometrically using electrodes of the invention.

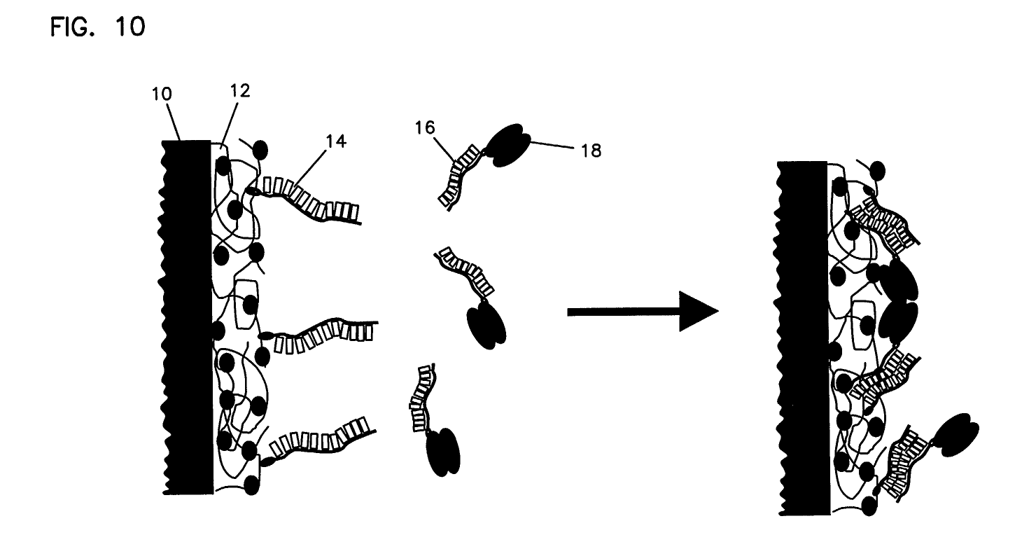

A schematic representation of the electrode structure is shown in FIG. 10. A 7 μm diameter carbon microelectrode 10 was coated with redox polymer 12 using the methods described above for Example 1, by electrophoretic deposition at constant potential in low ionic strength solution on the carbon microelectrode. A single-stranded probe oligonucleotide (sensor oligonucleotide) 14 [SEQ. ID NO: 4] charged with a reactive methyl imidazole group (carbondiimide-activated) was electrophoretically deposited and covalently attached to the redox polymer film 12 as described above for Example 2, to form the working electrode. The target single- stranded oligonuclotide (complementary or with one [SEQ. ID NO. 2] or 4 [SEQ. ID NO. 3] base-pair mismatch) was covalently bound to thermostable soybean peroxidase 18 to form the SBP-labeled target sequence. The working electrode and target oligonucleotides were then reacted at varying hybridization temperatures.

With hybridization of the target and probe oligonucleotides, the peroxidase is brought into close contact with the redox polymer and the redox polymer film becomes a catalyst for H₂O₂ electroreduction at 0.06V vs Ag/AgCl. A catalytic current was measured, and the current observed corresponded to that generated by approximately 40,000 surface-bound and electrically connected soybean peroxidase molecules.

This oligonucleotide sensor was capable of detecting hybridization of the 18 base pair oligonucleotide probe shown in the table below in real time, e.g., in less than 10 minutes.

By controlling the hybridization conditions, that is, by controlling the hybridization temperature, the sensor was able to discriminate between oglionucleotides having a single base pair mismatch, making the sensor of the invention useful in small arrays of the microelectrodes for DNA sequence applications and diagnosis of genetic diseases.

!? SEQ. ID.? ? ? !SEQUENCE? NO.? Tₘ

| | | | |
|---|---|---|---|
| Complementary | 5′-GAA ACA CCA ATG ATA TTT 3′ | 1 | 59.5°C. |
| Target | | | |
| Single BP | 5′GAA ACA CCA GTG ATA TTT 3′ | 2 | 54.5°C. |
| Mismatch | | | |
| Four BP | 5′GAA ACA CCA AAG ATA GATA3′ | 3 | 39.5°C. |
| Mismatch | | | |
| Probe Sequence | 3′CTT TGT GGT TAC TAT AAA 5′ | 4 | - |

The melting point for the complementary strand [SEQ. ID NO: 1] shown in the table above was determined by the following equation:

Tm=81.5°−16.6(log₁₀[Na])+0.41(%6+C)−600/(length in base pairs).

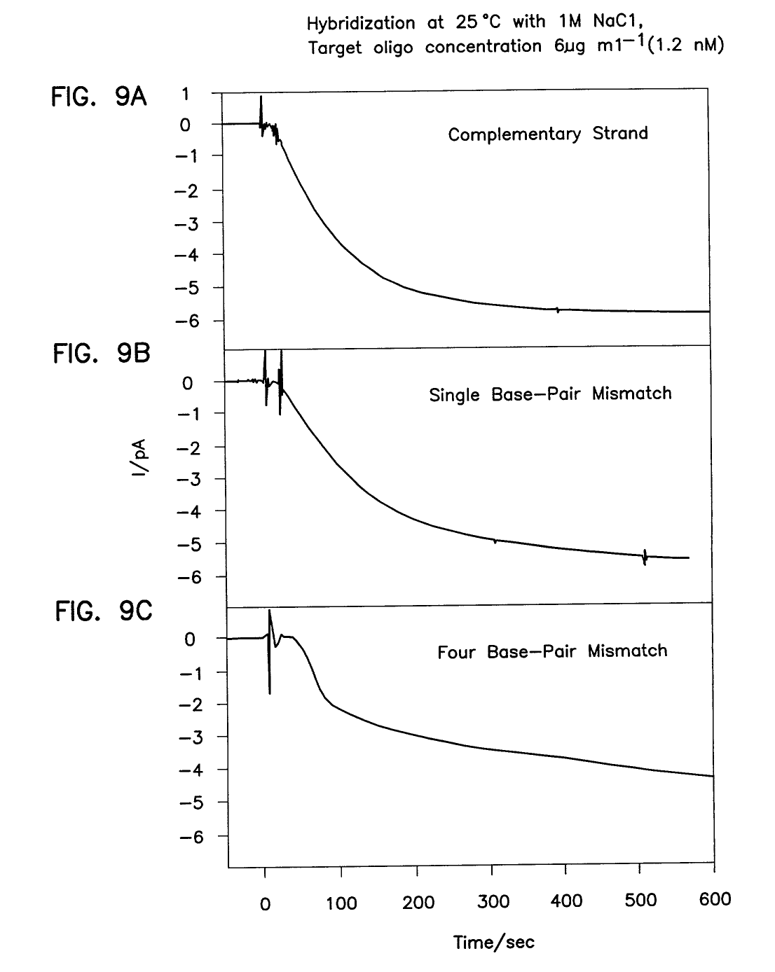

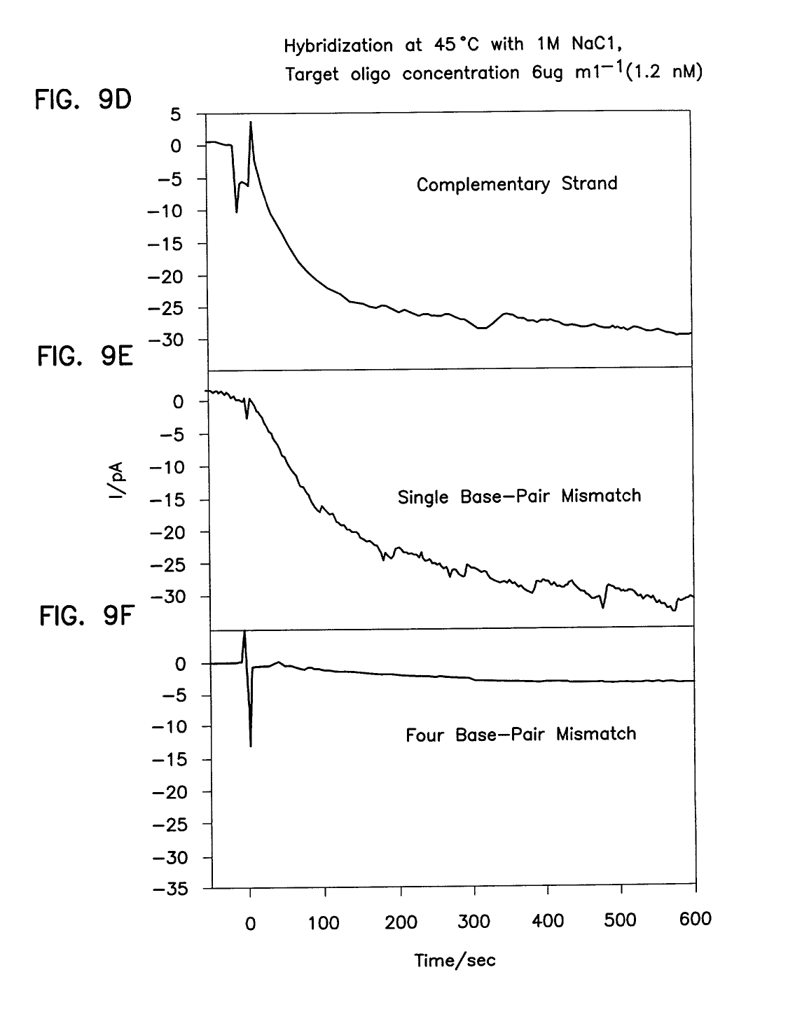

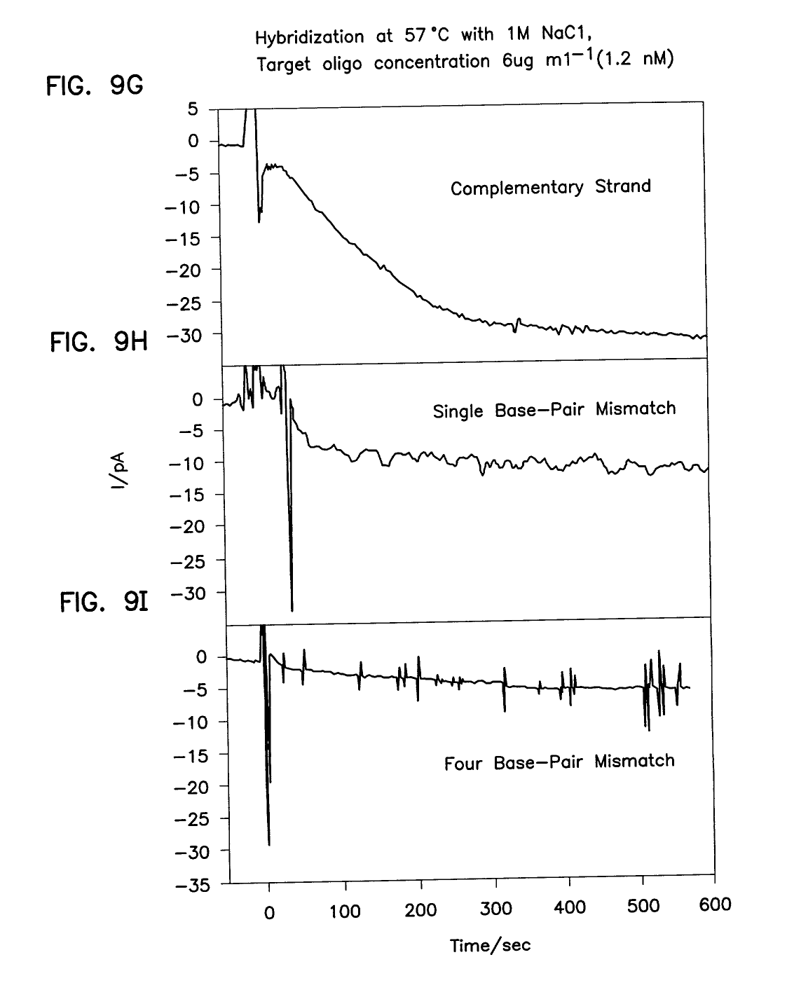

The melting point is reduced for a single base pair mismatch by about 5° C., and lowered a further 15° C. with four mismatches. As shown in FIGS. 9A-9C, at 25° C. all three oligoncleotides hybridized with the target probe, including the four base pair mismatch. At 45° C., the four base pair mismatch did not hybridize, the hybridization temperature being above its melting point of about 40° C. (FIGS. 9D-9F). At 57° C., which hybridization temperature is above the melting point for the single base pair mismatch but below that of the complementary strand, only the complementary strand was hybridized (FIGS. 9G-9I).

Thus, the sensor and assay system of the invention is able to discriminate between complementary and single base pair mismatch oligonucleotides by controlling the stringency of hybridization, e.g., the reaction temperature.

The number of copies measured is estimated to be about 40,000 molecules of target at a single electrode.

### Example 7

### Electochemical Array and System for the Detection of Hybridization Events

Unless specifically noted, the materials, methods and equipment used were the same as described above for Example 6.

All glassware was washed by soaking in Aquet (VWR) overnight then rinsed with deionized water. Sodium periodate (cat#31,144-8), 1-(3-dimethylaminopropyl)-3-ethylcarbodiimide hydrochloride (EDC) (cat # 16,146-2), were purchased from Aldrich. Imidazole (cat #I-20-2) and soybean peroxidase (SBP) (cat# P-1432) were purchased from Sigma. All buffer salts and other inorganic chemicals were obtained from Sigma or Aldrich. The electron-conducting redox polymer, (PAA-PVI-Os), a 7:1 co-polymer of acrylamide, acrylhydrazide, and 1-vinylimidazole, the imidazole functions complexed with [Os(4,4′-dimethyl-2,2′-bipyridine)₂C1]^{+/+2}, was synthesized as previously described.

The probe and target oligonucleotides used were purchased from Genosys and are listed in Table 1. The 18-base probe was purchased with a 12-T spacer and the 18-base targets with 12-C spacers between their oligonucleotide and terminal primary amine-functions. The primary amines were used to form Schiff bases with aldehyde-functions of periodate-oxidized oligosaccharides of SBP, which were subsequently NaBH₄ reduced to secondary amines.

The current-time traces were recorded with a Y-t recorder (Kipp&Zonen, Holland). Electrochemical measurements were carried out using a low noise CH Instruments model 832 electrochemical detector in conjunction with a Pentium computer. The water-jacketed cell was placed in a grounded Faraday cage. Unless otherwise stated the three-electrode system used consisted of the microelectrode, a Ag/AgCl reference electrode and a large area platinum flag or wire. For measurements above 25° C. an agar salt bridge was used to maintain the reference electrode at a constant temperature of 25° C. Microelectrodes were built in house by sealing individual 7 μm diameter carbon fibers (Goodfellow, UK) in soft glass tubes (Kimble Products, USA.) in a butane flame. The electrode surface was exposed by fracturing the glass and polishing, first with sand paper then with aluminas of decreasing particle size to 0.3 μm. The electrodes were tested by cyclic voltammetry in ferrocenemethanol and in pH 7.0 phosphate buffer solution for absence of leaks and for perfection of the glass seal of the carbon fiber. The repolished microelectrodes were stored in deionized water.

Polymer Deposition:

In the miniature cell that was designed for the electrophoretic deposition, the counter-electrode was a 100 μm thick platinum foil serving as the base of the cell. A silver wire served as a pseudo-reference electrode. 100 μl of a 0.085 mg ML⁻¹ PAA-PVI-Os redox polymer solution in deionized water was placed in the cell and was used for up to 25 depositions before being replaced.

After the microelectrode was connected to the potentiostat, it was lowered into the redox polymer solution, using a micro-manipulator, until the tip of the microelectrode was 1 mm from the counter electrode at the base of the cell. The redox polymer was electrophoretically deposited by poising the potential of the microelectrode at −1.025V (Ag/AgCl) for 2 minutes, after which the electrode was washed with de-ionized water. The deposition was then confirmed by cyclic voltammetry in buffer solution. It was essential that neither the working electrode nor its contact nor any part of the cell be touched, as the buildup of static electricity could change the electrochemical characteristics of the redox polymer coating of the microelectrode.

Probe Oligonucleotide Attachment:

A solution of 450 to 550 μg of the probe oligonucleotide in 50 μl of pH 7, 20 mM methyl imidazole buffer, was added to 50 μl of 0.2 M EDC in the same buffer. This mixture was kept overnight at 4° C. The solution was then diluted with 450 μl of water and the volume was reduced to 50 μl using a Microcon tube (Amicon) with a 3000 dalton cut off membrane. This procedure was repeated twice to remove the buffer salts from the solution. The solution was reduced to 50 μl, then transferred to an electrochemical cell similar to the one described above for the redox polymer deposition and used for the electrophoretic deposition of the probe oligonucleotide. Because the oligonucleotide was EDC-activated, it reacted with hydrazide functions of the redox polymer on the microelectrode. The deposition conditions of the EDC-activated oligonucleotide were similar to those of the deposition of the redox polymer except that the applied potential was +0.9 V (Ag/AgCl) and the duration was 5 minutes, unless otherwise stated. After the oligonucleotide was deposited a cyclic voltammogram was recorded in buffer solution containing 1 M NaCl.

Labeling of Oligonucleotides with SBP:

The three 18-base target oligonucleotides of Table 1, one perfectly complementary to the probe [SEQ. ID NO: 1]; one with a single base mismatch [SEQ. ID NO: 2]; and one with four mismatched bases [SEQ. ID NO: 3]were purchased with 5′-amine-terminated 12-carbon spacers. They were labeled with SBP as follows: 10 mg SBP was dissolved in 0.25 mL pH 7, 0.1 M phosphate buffer and 0.25 mL of freshly dissolved sodium periodate in water was added. The solution was left to stand for 1 hour in the dark, then passed through a standard G-25 gel filtration column (60 cm long, 1.5 cm diameter). The concentration of the resulting oxidized enzyme was determined by the Biorad protein assay (Protein assay kit II). A ten fold molar excess of enzyme was added to between 200 and 300 μg of oligonucleotide bringing the volume to 0.5 mL. The solution was allowed to react for 3 hours before 0.5 mL of 0.4 M NABH₄ was added, then left for 13 hours at 4° C. The resulting labeled oligonucleotide concentration was between 35 and 50 mM.

The activities of the SBP labels of the oligonucleotides were derived from the measured protein concentration and the rate at which the SBP catalyzed H₂O₂ oxidation of the leuco-dye 2,2′-azino-bis(3-ethylbenzthiazoline-6-sulfuric acid) (ABTS) to the dye. The initial rate of dye formation was measured spectrophotometrically, using a Hewlett Packard diode array UV/Vis, model 8452A spectrophotometer. The procedure involved adding 2.9 mL of 5 mM ABTS to 50 μl of peroxidase solution followed by 50 μl of 60 mM H₂O₂ to initiate the reaction. The change in absorbance at 404 nm was recorded for 60 seconds.

The specific activity of the native SBP was, as reported, 45% of the specific activity of native horseradish peroxidase at 25° C. (SIGMA catalog, 1997, page 812). After attachment of the SBP to the oligonucleotide, 57% of the activity was conserved. The loss of activity was caused by the periodate oxidation step, not the NaBH₄ reduction step. The isoelectric points were measured by isoelectric focusing electrophoresis (Phastgel System, Pharmacia). The isoelectric points were 9.1 for the native enzyme, 4.5 for the periodate-oxidized enzyme and 8.0 for the periodate oxidized then NaBH₄-reduced enzyme.

Amperometric Detection of Hybridization:

Hybridization was carried out in 1 mL of pH 7 HEPES (N-[2-hydroxyethyl]piperazine-N′-[2-ethanesulfonic acid]) buffer containing 1 M NaCl, 1 mM H₂O₂and 1 mM EDTA (ethylenediaminetetraacetic acid). The buffer solution was thermostatted at the stated temperature and was stirred using a rotating glass paddle powered by an air-powered motor. The electrode was connected to the potentiostat and polarized at −0.06V vs. Ag/AgCl. After 20 seconds the current stabilized between −5 to 15 pA and 10 μl of the oligo-SBP conjugate was injected into the 1 mL cell to bring the concentration to 0.4 nM. The change in the current with time was recorded, and the observed hybridization transients were fitted to a diffusion limited Langmuir equation using Sigma Plot graphing software (SPSS).

To determine the current reached when the redox polymer pre-coated electrodes were densely coated with SBP, the periodate oxidized enzyme was allowed to self-crosslink on the redox-polymer films of the microelectrodes. The crosslinking resulted of the condensation of aldehyde functions of the oxidized oligosaccharide of the enzyme with its surface (lysine and arginine) amines. Because in these experiments the redox polymer did not have hydrazide functions, covalent linking of the redox polymer and the oxidized enzyme was not the cause of SBP immobilization.

In a second experiment the electrode was pre-coated with the hydrazide-functionalized redox polymer. The EDC activated probe oligonucleotide was bound to the redox polymer through reactive electrophoretic deposition. The periodate oxidized SBP was then allowed to bind and self-crosslink on the electrode. These experiments were carried out in the hybridization buffer containing 0.4 nM of periodate oxidized SBP.

Results.

When the periodate-oxidized SBP was allowed to self-crosslink on the electrode coated with the redox polymer without reactive hydrazide functions, the H₂O₂ electroreduction current at 25° C. increased from nil to −45 pA then leveled off. The current leveled off at 30 pA when the electrode was coated with the hydrazide-functionalized redox polymer, then reacted with the electrophoretically deposited EDC-activated probe. No catalytic current was detected after the oxidized SBP was reduced with NaBH₄, with or without the oligonucleotide attached, and also if not initially reacted with periodate, showing that nonspecific adsorption of SBP to the redox polymer was not significant.

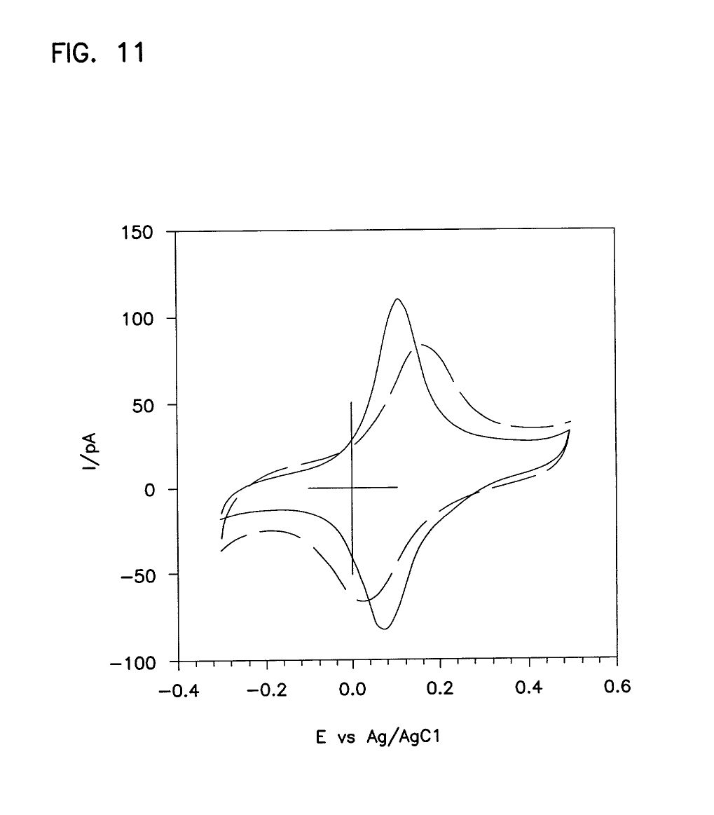

The solid curve of FIG. 11 is a typical voltammogram of the electrophoretically deposited redox polymer on the 7 μm microelectrode. The permeability of the redox polymer film to small molecules was probed using ferrocenemethanol. The permeability was so high, that the diffusion limited current at 0.5 V was only 2-5% below that of the uncoated electrode. To test the reproducibility of the coatings formed by electrophoretic deposition, the same electrode was re-polished and re-coated using the same redox polymer solution 25 times. The peak heights of the cyclic voltammograms of the 25 films were identical, the standard deviation in their heights being ±5%.

The deposition of the EDC-activated probe oligonucleotide onto the redox polymer resulted in the coupling of the reactive 5′ phosphate with hydrazide functions of the redox polymer. The reactive electrophoretic deposition of the probe increased the separation of the anodic and cathodic peaks (FIG. 11, dashed line). The integrals of the anodic and cathodic waves did not change, indicating that no redox polymer was lost as a result of the oligonucleotide deposition.

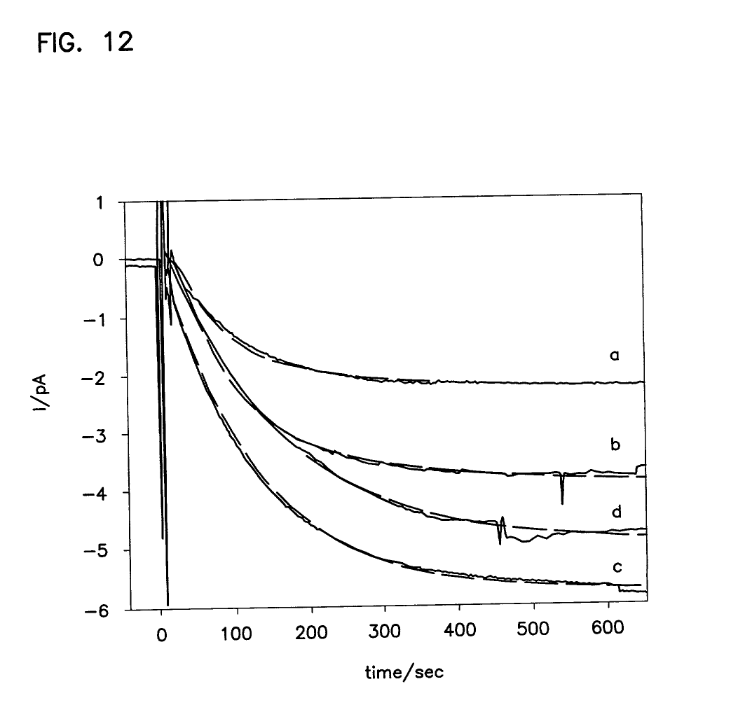

The effect of the amount, i.e. loading, of the EDC-activated probe oligonucleotide on the rate of target-hybridization and on the resulting H₂O₂ electroreduction current was studied. As seen in FIG. 12, optimal probe-loading was reached when the duration of its electrophoretic deposition was 5 minutes. When the probe deposition was continued for 10 minutes, the rise in current following introduction of the SBP-labeled target was slowed and the maximum current was reduced. For deposition times shorter than 5 minutes, the rise in current was not faster, but the maximum current was reduced.

When redox polymer without reactive hydrazide functions on the backbone was deposited in the same way, the current observed after 5 minutes hybridization of the SBP-labeled target oligonucleotide with the film, which was coated by 5 minutes electrophoresis of the probe oligonucleotide, was only 0.35-0.75 pA at 25° C.

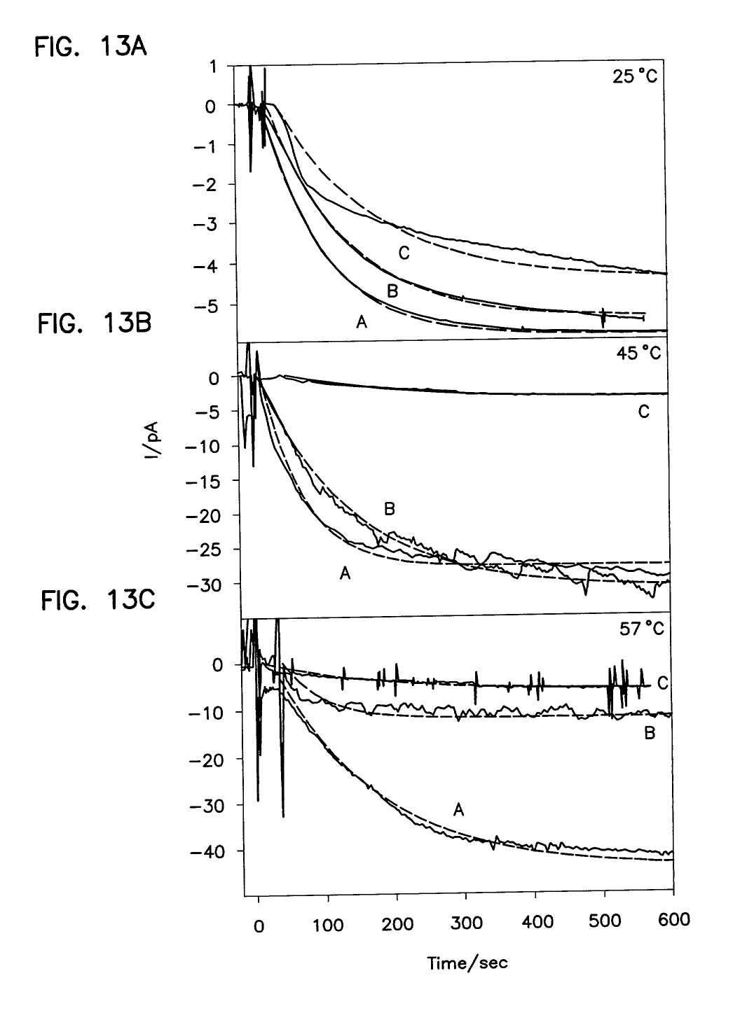

FIGS. 13A-13C show the current evolution at 25° C. (FIG. 13A), 45° C. (FIG. 13B), and 57° C. (FIG. 13C) for the perfect (curve A), single base-mismatched (curve B), and four base-mismatched (curve C) SBP target oligonucleotides listed in Table 1. At 25° C., H₂O₂ was electroreduced on electrodes with any of the three SBP-labeled targets. At 45° C., H₂O₂ was electroreduced only when the target was perfectly matched or when only one of its bases was mismatched, but not when four of the bases were mismatched. At 57° C., H₂O₂ was electroreduced efficiently (45 pA current) only when the target was perfectly matched. The current dropped to 12 pA when a single base was mismatched and to 6 pA when four base-pair were mismatched. The currents for the perfectly matched hybrid at 25° C., 45° C. and 57° C. were, respectively, 6 pA, 28 pA and 45 pA.

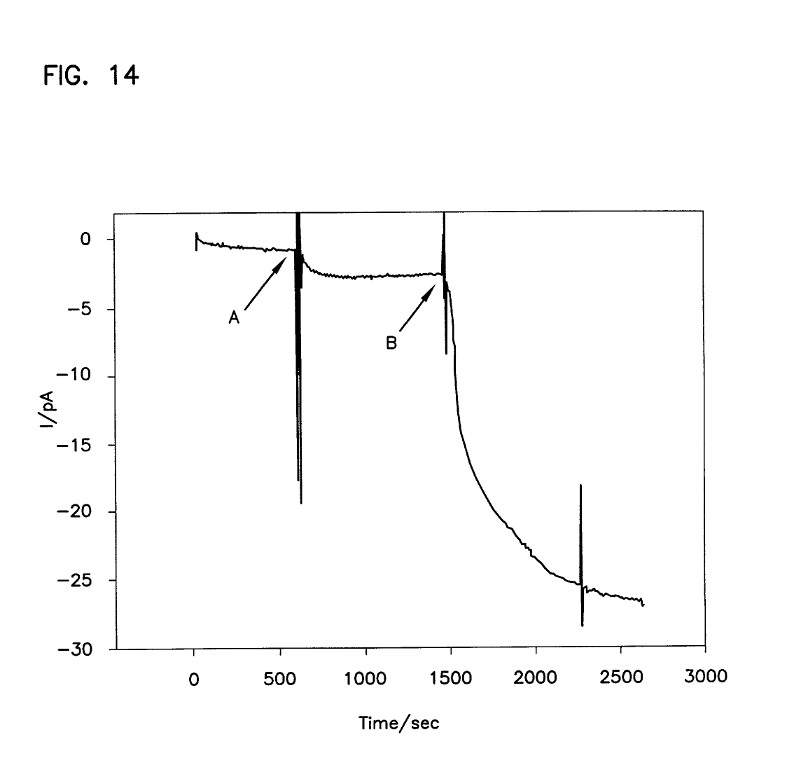

In FIG. 14 results of hybridization at 45° C. are shown using SBP-labeled target with 4-mismatched bases. The 4-base mismatch target (10 μl of 40 μm SBP) was introduced, at t-550 seconds (Arrow A) followed by introduction of the fully complementary SBP-labeled target (10 μl of 40 μM SPB) at t=1450 seconds (Arrow B). The respective attained currents were 2.3 pA and 25 pA.

Interpretation of the Results

A scheme for detecting a hybridized probe is shown in FIG. 15A. A redox polymer film was electrophoretically deposited on the electrode. This film was reacted, in a second electrophoretic deposition step, with the 5′-activated-poly-T spacer of the oligonucleotide probe. Upon hybridization with the SBP-labeled target oligonucleotide, electrons were flowing from the electrode through the redox polymer to heme-centers of the label, reducing these centers, as diagramed in FIG. 15B. SBP is preferred over the more active horseradish peroxidase for labeling the target because of its superior thermal stability (see, for example, Vreeke et al., 1995 Anal. Chem. 67: 4247).

Because electrophoretic deposition was restricted to the conductive area of the glass-embedded carbon fibers, redox polymer films of reproducible dimensions and characteristics were deposited when the electrode and solution were also reproducible. This is important because, when multiple electrodes are used in arrays, then differences between the currents of electrode-pairs can be measured and the significance of small differences in current will depend on the reproducibility of the coatings. Dimensions of electrodes can be reproduced, when made by the processes used in the manufacture of microelectronic circuits, within ±0.05 μm. In 25 successively deposited films the voltammetric peaks showed a normal distribution, with the standard deviation, σ, being ±5%.

The purpose of pre-coating the microelectrodes with a thin layer of redox polymer was to make the electrical contact, between the reaction centers of SBP and the electrode independent of the orientation of the target-labelling-SBP. In absence of a redox polymer film, electrical contact is established only with those SBP heme centers that are near the electrode surface. For horseradish peroxidase on vitreous carbon the fraction of properly oriented enzyme molecules is only about 1%. When the electrode was coated with a redox polymer, its redox potential reducing relative to the potential of the peroxidase, then electrons could flow to heme centers of the peroxidase irrespective of orientation. The electrons were now transferred through collisions of heme-centers with randomly moving segments of the crosslinked redox polymer film. The mobility of these tethered segments increased when the redox polymer was hydrated. Electrons diffused through the redox polymer by the related process of collisional self-exchange between approaching redox function-carrying segments of the polymer network. FIG. 15B shows the resulting scheme of electron-transport between the electrodes and the SBP labels of the targets. Such transport results in the catalysis of the electroreduction of H₂O₂ to water (Equation 1) at −0.06V (Ag/AgCl). At this potential H₂O₂ is not electroreduced on carbon in absence of the catalyst

H₂O₂+2 e−+2H⁺−>2H₂O (1)

Because of the fast electron exchange and because the redox polymer was well adsorbed on the electrode, the peaks of the anodic and cathodic waves of the voltammogram of the redox-polymer coated microelectrodes were separated only by about 20 mV (FIG. 11, solid curve). The peak separation was increased to 180 mV after the probe oligonucleotide is bound to the redox polymer (FIG. 11, dashed curve), indicative of much slower electron transport. The apparent cause of the sluggish transport was the formation of ion bridges between the polyanionic probe and the polycationic redox polymer, which restricted the segmental mobility of the redox polymer and thereby the frequency of electron transferring collisions.

FIG. 11 shows that overloading of the redox polymer film with the probe oligonucleotide reduced the current after the SBP-labeled hybrid was formed. The current increased initially (FIG. 12, curves a, b, and c) as the amount of probe was built up and more of the SBP-labeled target was captured. However, when the amount of probe was excessively increased the current decreased and the rate of hybridization, evidenced by the rate of change in current, also slowed (FIG. 12, curve d). A possible cause of the reduction in current was the restriction of the movement of segments of the redox polymer upon excessive ion-bridging, compounded by dilution of the density of electron-exchanging redox centers.

Consistently, the current was reduced by 30% upon incorporation of probe oligonucteotide in the redox polymer film not only when the current was flowing through the SBP-label of the hybrid, but also in absence of hybridization, when periodate-oxidized SBP was allowed to self-crosslink on the film. The sluggish increase in current when the redox polymer film was overloaded with the probe is also attributed to the formation of ion-bridges. These make the film rigid, slowing the access of the SBP-labeled target to probe oligonucleotides.

The optimal duration of the deposition process was 5 minutes. The time dependence of the current, which represents the rate of hybridization, was found to be well described (R≈0.99) by the diffusion limited Langmuir equation (Equation 2) (Peterlinz et al., 1996, Langmuir, 12:4731).

e=eₘₐₓ[1−exp(−k_{D}t^{½})] Equation 2

In the equation k_{D} is the surface binding rate (here the rate of hybridization of the SBPlabeled target to the electrode-bound probe) which is related to the rate of diffusional mass transport and eₘₐₓ is the maximal surface concentration of enzyme, reached after hybridization of all possible probes that can hybridize. For a thin redox polymer film and neglecting diffusion of the substrate (H₂O₂ in the present case) the saturation current is given by Equation 3.

$${I}_{cat} = \frac{2 {Fak}_{{cat}^{e}}}{1 + \frac{{k}_{cat}}{k [ {Os}^{2 +} ]} + \frac{{K}_{M}}{[ S ]}} \qquad \text{Equation 3}$$

In equation 3, k is the rate of the reaction between the mediator and enzyme, [OS²⁺] is the concentration of the reduced redox centers in the film; K_{M} and k_{cat}, have their usual meanings, and [S] is the substrate (H₂O₂) concentration. Because in the experiments the substrate concentration was much higher than K_{M}, the third term in the denominator of Equation 3 could be neglected. Therefore the current was limited either by electron diffusion in the film or by the enzyme's turnover rate. Through combining Equation 2 with the simplified Equation 3, Equation 4 was derived. This equation fitted the curves of FIGS. 2 and 3.

I_{cat}=b[1−exp(−k_{D}t^{½})] Equation 4

$$In Equation 4 , b = \frac{2 k [ {Os}^{2 +} ] {FAk}_{{cat}^{e} max}}{k [ {Os}^{2 +} ] + {k}_{cat}}$$

The best fit parameters for the hybridization transients of FIG. 10 are summarized in Table 2, which shows the best fit parameters to Equation 4 for microelectrodes with different probe loadings. The calculated and the measured (FIG. 2) currents fitted equation 4 with a mean difference of 2% or less in any of the experiments.

TABLE 2

| Oligonucleotide Deposition time | | lb/pA | kD/sec-1 | R2 |
|---|---|---|---|---|
| 1 | minute | −2.26 | 0.089 | 0.99 |
| 2.5 | minutes | −3.86 | 0.093 | 0.99 |
| 5 | minutes | −5.79 | 0.0951 | 0.99 |
| 10 | minutes | −4.6 | 0.077 | 0.99 |

While the k_{d} values for films a, b and c, that were not overloaded with the probe were similar, k_{d} of the overloaded film was significantly lower, suggesting that the hybridization-causing diffusive step was restricted in the matrix with the excessive amount of oligonucleotide. The time dependence of the current fitted the diffusion limited Langmuir equation also when the hybridization was carried out at different temperatures and with mismatched bases in the oligonucleotides. However, the noise increased with the temperature and R² was reduced.

The best fit parameters to the hybridization transients for the one fully and the two imperfectly matched oligonucleotide sequences are listed in Table 3, which shows the best-fit parameters to Equation 4 for the three targets at 25°, 45°, and 57° C., shown in FIG. 13. The calculated and the measured (FIG. 12) currents fitted equation 4 with a mean difference of 2% or less in the experiments at 25° C.; 4% at 45° C.; and 10% at 57° C.

TABLE 3

| Temperature | Target oligonucleotides | b/pA | kD/sec-1 | oc |
|---|---|---|---|---|
| 25° C. | Complementary | −5.74 | 0.096 | 0.99 |
| | Single base mismatched | −5.48 | 0.098 | 0.95 |
| | Four bases mismatched | −4.46 | 0.084 | 0.76 |
| | Complementary | −27.7 | 0.117 | 0.99 |
| 45° C. | Single base mismatched | −30.2 | 0.086 | 0.89 |
| | Four bases mismatched | −3.44 | 0.077 | 0.97 |
| 57° C. | Complementary | −44.6 | 0.082 | 0.91 |
| | Single base mismatched | −11.8 | 0.133 | 0.56 |
| | Four bases mismatched | −5.9 | 0.079 | 0.66 |

The dashed curves in FIG. 12 represent the equation fitted with the constants listed in Table 3. The diffusion limited Langmuir equation fitted these transients particularly well, especially in the case of the complementary oligonucleotide at 25° C. shown in FIG. 10. In some cases the fitting was made difficult by the noise and in one case, that of the four base pair mismatch at 25° C., the fit was poor.

The increase in the currents for the perfectly matched hybrids (25° C., 6 pA; 45° C., 28 pA; and 57° C., 45 pA) with temperature, yields an activation energy of 60 kJ mol⁻¹, similar to activation energy reported for a related redox polymer (Gregg et al., 1991, J. Phys. Chem., 95:5970). Because the SBP-label contacted electrically the redox polymer only below the melting temperature of the hybrid, the activation energy activated currents at 25° C., 45° C., and 57° C. differed significantly when the hybrids were perfectly matched, had a single basepair mismatch, or contained four mismatched base pairs. The theoretically estimated melting temperature of the 18-base hybrid with a single mismatched base pair is approximately 5-7° C. below that of the perfect hybrid when the mismatch is in the middle of the oligonucleotide and the mismatched base pair is GC (Anderson, 1995, in: Gene Probes 2, A Practical Approach, Hames and Higgins, Eds., Oxford University Press, Inc., New York, pages 1-29.) For the four base-pair mismatch the theoretically estimated melting temperature is 20-23° C. below that of the perfectly matched hybrid. The actual melting points of the 18-base pair hybrids when perfectly matched, mismatched in a single base pair and mismatched in four base pairs are 59.5° C., 54° C., and 37-40° C., respectively. Consequently, a current should flow in the case of the perfectly matched hybrid at any of the three temperatures, 25° C., 45° C. or 57° C. In the case of the hybrid with a single mismatched base pair a current should flow at 25° C. and at 45° C., but not at 57° C.; and in the case of the hybrid with four mismatched base pairs, a current should flow only at 25° C., not at 45° C., nor at 57° C. That this was indeed the case is seen in FIG. 13. For example, at 57° C. the current for the perfectly matched hybrid was 45 pA, while the current for the hybrid with a single mismatch was 13 pA. FIG. 12 shows the result form an experiment carried out at 45° C., where first the SBP-labeled target hybridizing below 37-40° C. with four mismatched bases was added, followed by the complementary SBP-labeled target, hybridizing at temperatures up to 59.5° C. This experiment showed that the presence of an extraneous oligonucleotides with a partially matching sequence does not interfere with the hybridization of the matched target, nor does it affect the magnitude of the current (about 30 pA) reached upon hybridization (FIGS. 13B and 14).

The number of copies producing the current was estimated from the turnover rate of the SBP label. The rate of turnover of the SBP label is 460 s⁻¹ at 25° C. With two electrons being transferred per turnover, this turnover rate corresponds to a current of 1.5×10⁻¹⁶ A per label. At 25° C. the saturating current measured upon complete hybridization was 5 pA, the output of the “wired” and active labels of 34,000 copies. For the 7 μm diameter electrode, the corresponding surface coverage was 1.4×10⁻¹³ moles cm⁻², which agrees well with the theoretically calculated surface density rang of 0.03 to 3.8×10⁻¹³ moles cm⁻²for a probe with 18 base pairs on a solid surface (Chan et. al., 1995, Biophys. J. 69:2243).

Conclusion

In summary a single-base mismatch in an 18 base oligonucleotide was amperometrically sensed with, and amplified by, a redox polymer coated microelectrode. The detected current was generated by about 40,000 active and “wired” copies of the thermostable SBP-labeled hybrid.

Using the method of the invention, the presence of a gene or RNA segment hybridized to an oligonucleotide bound to the redox polymer, would be queried, for example, with an SBP-labeled sequence hybridized to a different region of the gene. See, for example, FIG. 14.

### Example 8

### Electrochemical Detection of a Nucleic Acid Sequence

In the same manner as described in the Examples above, one or more specific hybridization probe oligonucleotide is immobilized onto an electrode, preferably onto an electrode array, and most preferably via covalent binding to redox polymer on the electrode. A test sample containing the target sequence to be detected is reacted with the immobilized oligonucleotides under appropriate hybridization conditions.

The test sample is also reacted with one or more second specific oligonucleotide probes, the second oligonucleotide probe being labeled with a catalyst, preferably with a thermostable peroxidase, and most preferably with soybean peroxidase. In the preferred system, reaction of the test sample with the immobilized first oligonucleotide and the second labeled oligonucleotide is simultaneous. Such a system is diagramatically represented in FIG. 15C and in FIG. 16.

Hybridization of both the first and second oligonucleotide probes to the target sequence results in the generation of a current at the working electrode, under the scheme described in FIG. 15B, which current is correlated with the hybridization event.

### Example 9

### A Diagnostic Array for Pathogen Screening

A sensor array for the screening of a patient sample for the presence of a pathogenic organism is produced by selectively depositing oligonucleotide probes diagnostic of a particular pathogen in an array using the methods described above. Specifically, a plurality of electrodes is deposited on a substrate. In a preferred embodiment, 100 or more electrodes are deposited to form the array. The deposited electrodes are coated with a redox polymer, as described above.

A series of oligonucleotide probes diagnostic of particular pathogens is coupled to the electrodes, one specific sequence per electrode. In a preferred embodiment, some redundancy is built into the array for improved accuracy. Specific coupling of an oligonucleotide to an electrode of the array is achieved via the electrophoretic deposition technique described above. The oligonucleotide attracted to the electrode is then coupled to the redox polymer via reactive groups. The electrophoretic deposition process is repeated with different oligonucleotide probes to form the diagnostic array.

Each oligonucleotide probe preferably contains about 8 to 100 bases, more preferably 10 to 40 bases, and most preferably, about 15 to 30 bases for hybridization to complementary sequence.

In the diagnostic assay, a patient sample is obtained, for example, blood, feces, or tissue swab or from water, air or food. The nucleic acid sequences may or may not be separated from the sample, tissue, or cells, but is preferably cleaved by restriction enzyme digestion, preferably by an enzyme having a short recognition site, e.g., 4 bases. The cleaved sample is then preferably denatured, for example by heating and rapidly cooling or by exposure to a solution of low ionic strength to separate single strands of DNA.

The denatured sample is applied to the array under conditions suitable for hybridization. Hybridization of sample DNA to a particular oligonucleotide probe is detected electrochemically at the electrode. In a preferred embodiment, a second specific oligonucleotide probe, which hybridizes to a different region of the digested DNA, is added, the second probe labeled, for example, with a catalyst. The catalyst is preferably a redox enzyme, and is most preferably a thermostable redox enzyme such as soybean peroxidase. Upon hybridization of a sample nucleic acid sequence to both the first and second diagnostic oligonucleotide probes, the enzyme makes electrical contact with the wiring redox polymer (e.g., hydrogel) and the electroreduction of hydrogen peroxide is catalyzed. The current generated at the electrode sensor is diagnostic of the particular pathogen.

This specification contains numerous citations to publications and patents, each of which is hereby incorporated by reference as if fully set forth.

1. A nucleic acid sensor for detecting target nucleic acid, the nucleic acid sensor comprising:
a) an electrode;
b) redox polymer disposed on the electrode;
c) enzyme disposed on the electrode; and
d) a sensor nucleic acid coupled to the redox polymer,
wherein, in the presence of a substrate, the enzyme generates a detection compound, and wherein binding of the sensor nucleic acid to the target nucleic acid results in an increased rate of oxidation or reduction of the detection compound.

2. The nucleic acid sensor according to claim 1 wherein the redox polymer comprises a redox hydrogel.

3. The nucleic acid sensor according to claim 1 wherein the enzyme is immobilized in the redox polymer.

4. The nucleic acid sensor according to claim 1 wherein the enzyme generates hydrogen peroxide as the detection compound.

5. The nucleic acid sensor according to claim 1 wherein the enzyme is choline oxidase, hydroxylase, or hydrolase.

6. The nucleic acid sensor according to claim 1 further comprising the substrate.

7. An array comprising a plurality of electrically isolated nucleic acid sensors of claim 1 disposed on a substrate.

8. The array according to claim 7 wherein the sensor nucleic acids of at least two of the nucleic acid sensors are different.

9. An array comprising:
a) a plurality of electrically isolated nucleic acid sensors, each nucleic acid sensor comprising:
(i) an electrode;
(ii) redox polymer disposed on the electrode;
(iii) enzyme disposed on the electrode; and
(iv) a sensor nucleic acid coupled to the redox polymer; and
b) one or more flow channels disposed on the array, each flow channel having a width of 200 μm or less,
wherein, in the presence of a substrate, the enzyme generates a detection compound, and wherein binding of the sensor nucleic acid to the target nucleic acid results in an increased rate of oxidation or reduction of the detection compound.

10. The array according to claim 9 wherein the enzyme is immobilized in the redox polymer.

11. The array according to claim 9 wherein the sensor nucleic acids of at least two of the nucleic acid sensors are different.

12. A method for detecting target nucleic acid, the method comprising steps of:
a) providing an array comprising a plurality of electrically isolated nucleic acid sensors, wherein each nucleic acid sensor comprises:
(i) an electrode;
(ii) redox polymer disposed on the electrode;
(iii) enzyme, wherein, in the presence of a substrate, the enzyme generates a detection compound; and
(iv) a sensor nucleic acid coupled to the redox polymer;
b) contacting the array with the target nucleic acid under conditions suitable for hybridization of the target nucleic acid to the sensor nucleic acid of one or more of the nucleic acid sensors;
c) providing a substrate for the enzyme to generate a detection compound;
d) generating a current as a result of an increased rate of oxidation or reduction of the detection compound; and
e) correlating current generated at one or more electrodes with hybridization of the target nucleic acid with the sensor nucleic acid of the one or more electrodes.

13. The method according to claim 12 wherein the enzyme is disposed on the electrode.

14. The method according to claim 12 wherein the enzyme is immobilized in the redox polymer.

15. The method according to claim 12 wherein the current is generated by a catalyst coupled to the target nucleic acid.

16. The method according to claim 15 wherein the catalyst is a thermostable enzyme.

17. The method according to claim 15 wherein the catalyst is peroxidase.

18. The method according to claim 12 further comprising determining the nucleic acid sequence of the target nucleic acid.

19. The method according to claim 12 wherein the step of contacting the array with the target nucleic acid comprises controlling stringency of the hybridization of the target nucleic acid to the sensor nucleic acid using temperature.

20. The method according to claim 12 wherein the step of contacting the array with the target nucleic acid under conditions suitable for hybridization of the target nucleic acid to the sensor nucleic acid comprises contacting the array with the target nucleic acid at a temperature that is less than a melting temperature when the target nucleic acid and the sensor nucleic acid are not mismatched, and the temperature is greater than a melting temperature when the target nucleic acid and the sensor nucleic acid have at least four mismatches.

21. The method according to claim 12 wherein the step of contacting the array with the target nucleic acid under conditions suitable for hybridization of the target nucleic acid to the sensor nucleic acid comprises contacting the array with the target nucleic acid at a temperature that is less than a melting temperature when the target nucleic acid and the sensor nucleic acid are not mismatched, and the temperature is greater than a melting temperature when the target nucleic acid and the sensor nucleic acid have at least one mismatch.

22. The method according to claim 12 wherein the step of contacting the array with the target nucleic acid under conditions suitable for hybridization of the target nucleic acid to the sensor nucleic acid comprises contacting the array with the target nucleic acid at a temperature that is between a melting temperature when the target nucleic acid and the sensor nucleic acid are not mismatched and a temperature that is 5° C. less than the melting temperature.

23. The method according to claim 12 wherein the step of contacting the array with the target nucleic acid under conditions suitable for hybridization of the target nucleic acid to the sensor nucleic acid comprises contacting the array with the target nucleic acid at a temperature that is between a melting temperature when the target nucleic acid and the sensor nucleic acid are not mismatched and a temperature that is 20° C. less than the melting temperature.

24. The method according to claim 12 further comprising diagnosing a disease.

25. A method for detecting target nucleic acid comprising:
a) providing an array comprising a plurality of electrically isolated nucleic acid sensors, wherein each nucleic acid sensor comprises:
(i) an electrode;
(ii) redox polymer disposed on the electrode;
(iii) enzyme, wherein, in the presence of a substrate, the enzyme generates a detection compound; and
(iv) a sensor nucleic acid coupled to the redox polymer;
b) contacting the array with the target nucleic acid under conditions suitable for hybridization of the target nucleic acid to the sensor nucleic acid of one or more of the nucleic acid sensors;
c) providing a probe nucleic acid that is capable of hybridizing to the target nucleic acid, wherein the probe nucleic acid is coupled to a catalyst;
d) generating a current by allowing the catalyst to catalyze an electrochemical reaction of the detection compound upon hybridization of the sensor nucleic acid and the probe nucleic acid to the target nucleic acid; and
e) correlating the current generated at one or more electrodes with hybridization of the target nucleic acid with the sensor nucleic acid of the one or more electrodes and the probe nucleic acid.

26. The method according to claim 25 further comprising determining the nucleic acid sequence of the target nucleic acid.

27. The method according to claim 25 further comprising diagnosing a disease.

28. The method according to claim 25 wherein the catalyst is a thermostable enzyme.

29. The method according to claim 25 wherein the catalyst is peroxidase.

30. The method according to claim 25 wherein the enzyme is disposed on the electrode.

31. The method according to claim 30 wherein the enzyme is immobilized in the redox polymer.

32. The method according to claim 25 wherein the step of contacting the array with the target nucleic acid under conditions suitable for hybridization of the target nucleic acid to the sensor nucleic acid of one or more of the nucleic acid sensors is performed simultaneously with the step of providing a probe nucleic acid that is capable of hybridizing to the target nucleic acid.

33. A kit for detecting target nucleic acid comprising:
a) a nucleic acid sensor comprising:
(i) an electrode;
(ii) redox polymer disposed on the electrode;
(iii) enzyme, wherein, in the presence of a substrate, the enzyme generates a detection compound; and
(iv) a sensor nucleic acid coupled to the redox polymer; and
b) a probe nucleic acid, wherein the probe nucleic acid is coupled to a catalyst,
wherein the catalyst catalyzes an electrochemical reaction of the detection compound upon hybridization of the sensor nucleic acid and the probe nucleic acid to the target nucleic acid.

34. The kit according to claim 33 wherein the enzyme is disposed on the electrode.

35. The kit according to claim 34 wherein the enzyme is immobilized in the redox polymer.

36. The kit according to claim 33, wherein the nucleic acid sensor is one of a plurality of electrically isolated nucleic acid sensors of an array.

37. The kit according to claim 36 wherein each nucleic acid sensor comprises:
a) an electrode;
b) redox polymer disposed on the electrode;
c) enzyme, wherein, in the presence of a substrate, the enzyme generates a detection compound; and
d) a sensor nucleic acid coupled to the redox polymer.

38. The kit according to claim 37 wherein the enzyme is disposed on the electrode.

39. The kit according to claim 38 wherein the enzyme is immobilized in the redox polymer.

40. The kit according to claim 36 wherein the sensor nucleic acid of at least two of the nucleic acid sensors are different.

41. The kit according to claim 33 wherein the catalyst coupled to the probe nucleic acid comprises a thermostable enzyme.

42. The kit according to claim 33 wherein the catalyst is peroxidase, glucose oxidase, glucose dehydrogenase, lactose oxidase, or lactose dehydrogenase.

43. The kit according to claim 33 further comprising a substrate for the enzyme.

44. The kit according to claim 43 wherein the substrate is hydrogen peroxide, glucose, or choline.

45. A kit for detecting target nucleic acid comprising:
a) a nucleic acid sensor comprising:
(i) an electrode;
(ii) redox polymer disposed on the electrode; and
(iii) a sensor nucleic acid coupled to the redox polymer; and
b) a probe nucleic acid, wherein the probe nucleic acid is coupled to a thermostable enzyme,
wherein the thermostable enzyme catalyzes an electrochemical reaction of a detection compound upon hybridization of the sensor nucleic acid and the probe nucleic acid to the target nucleic acid.

46. The kit according to claim 45 wherein the nucleic acid sensor further comprises an enzyme, wherein, in the presence of a substrate, the enzyme generates the detection compound.

47. The kit according to claim 46 wherein the enzyme is disposed on the electrode.

48. The kit according to claim 47 wherein the enzyme is immobilized in the redox polymer.

49. A method of making a nucleic acid sensor comprising:
a) depositing an electrode on a substrate;
b) coating the electrode with a redox polymer and an enzyme that generates a detection compound in the presence of a substrate; and
c) selectively coupling a sensor nucleic acid to the electrode by electrophoretic deposition.

50. The method according to claim 49 wherein the step of coating the electrode with a redox polymer comprises electrophoretically depositing the redox polymer onto the electrode.

51. A method of making an array for detecting target nucleic acid, the method comprising steps of:
a) depositing a plurality of electrodes on a substrate;
b) coating the plurality of electrodes with a redox polymer and an enzyme that generates a detection compound in the presence of a substrate; and
c) selectively coupling a sensor nucleic acid to one or more of the electrodes by electrophoretic deposition.

52. The method according to claim 51 wherein the step of coating the plurality of electrodes with a redox polymer comprises electrophoretic deposition of the redox polymer onto the plurality of electrodes.
